(19) United States
(12) Patent Application Publication
(10) Pub. No.: US 2011/0229416 A1
(43) Pub. Date: Sep. 22, 2011

(54) AMPHIPHILIC MACROMOLECULES FOR SOLUBILIZING NANOCRYSTALS

(76) Inventors: Kathryn E. Uhrich, New Brunswick, NJ (US); Sarah M. Sparks, New Brunswick, NJ (US); Sandra Rosenthal, Nashville, TN (US); Michael Schrender, Headington (GB)

(21) Appl. No.: 13/018,102

(22) Filed: Jan. 31, 2011

Related U.S. Application Data
(60) Provisional application No. 61/299,915, filed on Jan. 29, 2010.

(51) Int. Cl. A61K 49/00; A61P 43/00; C08G 65/335; C08G 65/334; C08G 65/333; B82Y 5/00

(52) U.S. Cl. 424/9.6; 424/9.1; 525/50; 977/927

(57) ABSTRACT

Certain embodiments of the invention provide amphiphilic macromolecules and nanocrystals encapsulated by amphiphilic macromolecules. Additionally, certain embodiments of the invention also provide nanocrystals that are water-solubilzed using functionalized AMs capable of coordinating to the nanocrystal surface.

### RELATED APPLICATION

This application claims benefit of U.S. Provisional Application Ser. No. 61/299,915 filed on Jan. 29, 2010, which application is herein incorporated by reference.

### BACKGROUND

Fluorescence is a common, versatile technique used to quantitatively detect the presence of fluorescent species. Fluorescence is particularly useful in the biomedical imaging applications due to the non-destructive nature of the technique. Organic fluorophores or dyes, such as fluorescein and Texas red, are commonly employed to tag biologically relevant systems. However, organic fluorophores generally have narrow and weak spectra, poor photostability, short fluorescence lifetime and pH sensitivity, such that they are only useful for short experiments under controlled conditions. In addition, organic fluorophores are not suited for multiplexed experimentation, in which multiple fluorophors are excited and emit fluorescence simultaneously, because often their excitation wavelengths are not the same or their emission bands overlap too much to obtain useful information.

Fluorescent nanoparticles, such as semiconductor quantum dots (QDs), have several advantages over typical fluorophores including size-dependent optical properties that can be easily tuned and have enhanced photostability relative to organic fluorophores. The vast majority of nanoparticle imaging agents, currently evaluated for biological imaging, are formed of heavy metals, such as iron, titanium, and cadmium. The formulation of these heavy metals into nanoparticles yields particles with magnetic and fluorescent properties useful for imaging. However, fluorescent nanocrystals can be hydrophobic and are often cytotoxic due to their heavy metal composition. Therefore, new safe and efficient fluorescence systems, useful, e.g., for imaging, biosensing, and other applications are needed.

In summary, light emitting nanocrystals, e.g., quantum dots, have useful qualities, such as their use as biological probes. However, these nanocrystals can be cytotoxic. Accordingly, nanocrystals that are less cytotoxic are needed.

### Summary of Certain Embodiments of the Invention

Accordingly, certain embodiments of the invention provide an amphiphilic macromolecule of formula (I):

D-X—Y—Z—R₁ (I)

wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; R₁ is a polyether; R^{a} is a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alkyl.

Certain embodiments of the invention provide novel amphiphilic macromolecules (AMs) and nanocrystals encapsulated by amphiphilic macromolecules. In certain embodiments of the invention, nanocrystals are water-solubilzed using functionalized AMs capable of coordinating to the nanocrystal surface.

Certain embodiments of the invention provide an encapsulate comprising a nanocrystal surrounded or partially surrounded by a plurality of macromolecules of formula (I):

D-X—Y—Z—R₁ (I)

or residues thereof, wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; each R₁ is independently a polyether; each R^{a} is independently a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alkyl.

Certain embodiments of the invention provide an encapsulate formed by combining a) a plurality of macromolecules of formula (I):

D-X—Y—Z—R₁ (I)

wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; each R₁ is independently a polyether; each R^{a} is independently a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alky; b) a nanocrystal, and c) a solvent; and allowing the macromolecules and nanocrystals to form the encapsulate.

### BRIEF DESCRIPTION OF THE DRAWINGS

FIG. 1 is a schematic image of a) ligand exchange coating of semiconductor nanocrystals and b) ligand capping, or encapsulation, of semiconductor nanocrystals.

FIG. 2 is a schematic depicting the replacement of TOPO and octadecylphosphonic acid with the phosphonic acid-modified AM, 1pM.

FIG. 3 shows digital photographs of the WLNCs (1) dispersed in chloroform, (2) with 1cM in H₂O, (3) with 1pM in H₂O, (4) with 1cM in H₂O, (5) with 1pM in H₂O, and (6) dispersed in H₂O.

FIG. 4 shows digital photographs of the WLNCs (1) dispersed in chloroform, (2) with 1cM in H₂O, (3) with 1pM in H₂O, (4) with 1cM in H₂O, (5) with 1pM in H₂O, and (6) dispersed in H₂O after filtration with a 0.45 μm syringe filter.

FIG. 5 is a graphical representation of the percent transmittance for various WLNC samples from 200-650 nm.

FIG. 6 is a graphical representation of the hydrodynamic diameters of polymer/WLNC aggregates. In solutions where two distinct size distributions were present, the smaller size distribution is represented with a black bar while the larger distribution is represented in grey. The percentage of the observed size distributions are displayed directly above particle size (expressed in nm). Error bars represent peak widths.

FIG. 7 shows digital photographs of the WLNC solutions following excitation with a long wavelength UV lamp at 365 nm. (top) WLNC (1) dispersed in chloroform, (2) with 1cM in H₂O, (3) with 1pM in H₂O, (4) with 1cM in H₂O, (5) with 1pM in H₂O, and (6) dispersed in H₂O, (bottom) WLNC (1) dispersed in chloroform, (2) with 1cM in H₂O, (3) with 1pM in H₂O, (4) with 1cM in H₂O, (5) with 1pM in H₂O, and (6) dispersed in H₂O after filtration with a 0.45 μm syringe filter.

FIG. 8 is a graphical representation of the fluorescence emission spectra of the WLNC dispersed in the indicated solvent and, where applicable, solubilized by the indicated polymers.

FIG. 9 shows qualitative images of increasing polymer concentration on the WLNC fluorescence emission, (top) increasing ratio of 1pM:WLNC, (bottom) increasing ratio of 1cM:WLNC.

FIG. 10 is a graphical representation of the fluorescence emission spectra of 1pM-solubilized WLNCs at a ratio of 80:1 1pM:WLNC compared with the WLNCs in chloroform shows a decrease in the fluorescence emission intensity for 1pM:WLNC 80:1 after 500 nm, yielding predominantly blue emission.

FIG. 11 is a graphical representation of the storage stability of 1pM-solubilized WLNCs at varying storage conditions quantified by precent fluorescence intensity at 525 nm (compared with day 0) over two weeks.

FIG. 12 shows digital photographs of the WLNCs (1) dispersed in chloroform, (2) with the carboxylic acid-terminal AM in H₂O, (3) with the phosphonic acid (propyl)-terminal AM in H₂O, (4) with the phosphonic acid (butyl)-terminal AM in H₂O, (5) with the phosphate-terminal AM in H₂O, (6) with the sulfate-terminal AM in H₂O, (7) dispersed in H₂O, (8) with the primary amine-terminal AM in H₂O, (9) with the hydroxyl(ethyl)-terminal AM in H₂O, (10) with the hydroxyl(propyl)-terminal AM in H₂O, (11) with the thiol-terminal AM in H₂O, (12) with the NHS-terminal AM in H₂O, and (13) with the diisopropylphosphine-terminal AM in H₂O.

FIG. 13 is a graphical representation of the percent transmittance data at 363 nm and 485 nm for solutions of WLNCs dispersed in water utilizing the indicated functionalized AMs. For each category, the left bar represents data at 363 nm and the right bar represents data at 485 nm.

FIG. 14 is a graphical representation of the hydrodynamic diameters observed for water-soluble assemblies of WLNCs with the indicated functionalized AMs.

FIG. 15 shows digital photographs following excitation with a long wavelength UV lamp (excitation=365 nm) of WLNCs (1) dispersed in chloroform, (2) with the carboxylic acid-terminal AM in H₂O, (3) with the phosphonic acid (propyl)-terminal AM in H₂O, (4) with the phosphonic acid (butyl)-terminal AM in H₂O, (5) with the phosphate-terminal AM in H₂O, (6) with the sulfate-terminal AM in H₂O, (7) dispersed in H₂O, (8) with the primary amine-terminal AM in H₂O, (9) with the hydroxyl(ethyl)-terminal AM in H₂O, (10) with the hydroxyl (propyl)-terminal AM in H₂O, (11) with the thiol-terminal AM in H₂O, (12) with the NHS-terminal AM in H₂O, and (13) with the diisopropylphosphine-terminal AM in H₂O.

FIG. 16 is a graphical representation of the fluorescence emission spectra of WLNCs dispersed in the indicated solvent and, where applicable, solubilized by the indicated functionalized AMs at 464 and 525 nm.

### DETAILED DESCRIPTION

Nanoscale amphiphilic macromolecules (AMs) can include a hydrophobic portion, formed of mucic acid derivatized with aliphatic side chains, linked to a hydrophilic poly(ethylene glycol) (PEG). Above the critical micelle concentration (CMC ˜10⁻⁷M), these linear macromolecules form micelles that are 15-20 nm in diameter. These micelles are capable of imparting water solubility to insoluble compounds. In this case, water-insoluble nanocrystals, e.g., fluorescent nanocrystals, can be solubilized using AMs by encapsulation within the micelle and/or ligand exchange on the surface of the nanocrystal. These systems have the ability to retain their fluorescence properties and can be utilized for labeling and tracking applications or as biosensors. For example, white-light emitting nanocrystals can be water-solubilized using AMs and retain their ability to emit white light. Further, the white-light emitting AM-coated nanocrystals have the ability to act as biosensors through a change in color of fluorescence emission or brightness in the presence of specific biological stimuli.

The ability to maintain optical properties following surface modification is a big challenge in the field. As described herein, evidence has been generated to demonstrate that AMs are capable of providing a water-soluble coating for fluorescent nanocrystals, with minimal effect to the optical properties. Additionally, the ability to maintain light emission, e.g., white-light emission, in aqueous media following surface modification has, to our knowledge, never before been achieved. Further, the light emitting AM-coated nanocrystals may have the ability to act as biosensors through a change in color of fluorescence emission or brightness in the presence of specific biological stimuli.

It was surprising to be able to impart water solubility without significantly affecting optical properties, biocompatibility of the fluorescent nanocrystals, and ability to sense biological changes based upon a change in fluorescence properties. Additionally, the nanocrystals themselves are extremely bright fluorescing but are cytotoxic. When coated with AMs, they become less-cytotoxic, or biocompatibile. Finally, in the presence of specific biological stimuli, the white-light emitting AM-coated nanocrystals may have the ability to act as biosensors through a change in color of fluorescence emission or brightness.

Based upon a change in optics, change in brightness of white light fluorescence or fluorescent color, in the presence of specific stimuli, these AM-nanocrystal species will be useful as biosensors for multiple applications such as glucose monitoring in diabetics, detection of toxin levels, hemoglobin oxygenation levels.

Successful encapsulation and/or coating of white-light emitting nanocrystals with AMs has been demonstrated. It has also been confirmed the materials have low cytotoxicity.

### Polymers

A polymer is a macromolecule composed of simple, small organic or inorganic molecules covalently linked in a repeating fashion with, e.g., hundreds to thousands of repeats. Their high molecular weights give polymers unique properties compared to small molecules including higher viscosities, decreased solubility, and increased mechanical strengths, which can be fine-tuned based upon size (or number of repeat units) and chemical composition. These properties make polymers useful for a multitude of applications including clothing, rubber, and plastics, as well as many biomedical applications including the delivery of drugs and diagnostics.

### Polymer Micelles

Polymer micelles, which are dynamic assemblies of amphiphilic polymers, are one type of polymer therapeutic. They have many attractive features including the ability to achieve a nanoscale size and their inherent amphiphilicity. In general, materials on the nanoscale are attractive for biomedical applications as this size mimics that of many biological entities. This feature enhances the circulation time of the material as the nanomaterials are not immediately recognized as being foreign to biological systems. In addition to nanoscale size mimicking biological entities, the amphiphilicity of polymer micelles mimics the amphiphilicity of cell membranes, which enhances the permeability of the micelles. Polymer micelles can be composed of a variety of structural components. Polymer micelles are composed of unimers that have a hydrophobic block covalently bound to a hydrophilic block. To be classified as a polymer micelle, one or both of these blocks is a polymer. Some common hydrophobic polymers include subunits such as poly(propylene oxide) (PPO), poly(D,L-lactide-co-glycolide) (PLGA), and polycaprolactone while common hydrophilic polymers are poly(ethylene glycol) (PEG), poly(N-vinyl-2-pyrrolidone) (PVP), and poly(vinyl alcohol) (PVA). One of the most commonly used hydrophilic polymers is PEG because not only is it inexpensive, but PEG also has extremely low toxicity and is capable of efficient water solubilization while further increasing circulation time of the polymer micelle by being a “stealth carrier”; it is not recognized by biological systems as a foreign material and therefore no immune response is elicited. It also increases stability of micelles, both for storage and within the serum of the body, and aids in shielding cargo in the core from enzymatic degradation.

Three main types of amphiphilic unimers are known to self-assemble into polymer micelles: diblock copolymers, triblock copolymers and a lipophilic component conjugated to a hydrophilic polymer. When both components of a unimer are polymers, the resulting polymers are termed diblock or triblock copolymers. Poly(ε-caprolactone)-PEG and Pluronic® are examples of commercially available diblock and triblock copolymers, respectively, that self-assemble to form polymer micelles. The triblock copolymer Pluronic® is composed of poly(ethylene oxide) (PEO)-poly(propylene oxide), (PPO)—PEO. Alternatively, the hydrophobic component of polymer micelles can be composed of a lipophilic small molecule linked to a hydrophilic polymer. One example of this type of polymeric unimer is PEGylated cholesterol.

Polymer micelles self-assemble in aqueous media such that the hydrophobic components of the unimers form a hydrophobic core that is ‘protected’ from the hydrophilic media and solubilized by the hydrophilic components that form a hydrophilic corona. The hydrophobic core of the polymer micelle can then be used to water-solubilize hydrophobic materials based upon interactions with the hydrophobic domain of the polymer.

The combination of many features make polymer micelles particularly attractive as polymer therapeutics: their amphiphilicity allows them to permeate well through cell membranes, their nanoscale size increases circulation time, they generally have good shelf and in vivo stability, and depending on the structure the loading of hydrophobic drugs can be highly efficient. However, the use of polymeric micelles is often limited due to micellar instability, as the micelle is in constant equilibrium with the unimers. The assembly of polymeric micelles is controlled by the critical micelle concentration (CMC), or the lowest concentration at which micelles form. Micelles with lower CMCs (such as 100 nM as compared with 100 μM) are more stable as micelle systems at higher concentrations (i.e. there are less free unimers in solution) and, therefore, drug loading is more efficient.

### Nanoparticle Imaging Agents

Within the last decade, the use of fluorescent nanoparticles, such as semiconductor nanocrystals, for biomedical imaging applications has led to significant advances in fluorescence-based systems for imaging and tracking, as well as newer fluorescence-based techniques including biosensing, diagnostics and theragnostics (R. J. Martin-Palma, M. Manso, V. Tones-Costa, Sensors 2009, 9, 5149; A. F. E. Hezinger, J. Tebmar, A. Gopferich, European Journal of Pharmaceutics and Biopharmaceutics 2008, 68, 138). Nanoparticle imaging agents have been used for applications from cellular imaging and material tracking to disease diagnosis and treatment (K. T. Thurn, E. M. B. Brown, A. Wu, S. Vogt, B. Lai, J. Maser, T. Paunesku, G. E. Woloschak, Nanoscale Research Letters 2007, 2, 430). The vast majority of nanoparticle imaging agents currently evaluated for biological imaging are formed of heavy metals, such as iron, titanium, and cadmium. The formulation of these heavy metals into nanoparticles yields particles with magnetic and fluorescent properties useful for imaging.

Fluorescent nanoparticles, such as semiconductor quantum dots (QDs), have several advantages over typical fluorophores including size-dependent optical properties that can be easily tuned and have enhanced photostability relative to organic fluorophores. However, these nanoparticles are often water-insoluble and cytotoxic in nature.

### Solubilization of Nanoparticle Imaging Agents Using Polymers

As synthesized, the metal surfaces of many fluorescent nanoparticles are hydrophobic alkanes such as tri-octyl phosphine oxide (TOPO), hexadecylamine, or octadecylamine that render the nanocrystals hydrophobic (A. F. E. Hezinger, J. Tebmar, A. Gopferich, European Journal of Pharmaceutics and Biopharmaceutics 2008, 68, 138; N. I. Hammer, T. Emrick, M. D. Barnes, 2 2007). Additionally, due to their heavy metal composition, fluorescent nanocrystals are not biocompatible (R. J. Martin-Palma, M. Manso, V. Tones-Costa, Sensors 2009, 9, 5149). Thus, for use in biological applications, they should be modified to render them hydrophilic and biocompatible.

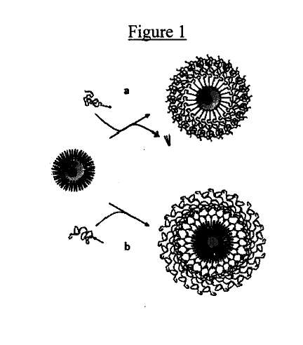

Two methods to induce hydrophilicity and biocompatibility to QDs are encapsulation and ligand exchange (A. F. E. Hezinger, J. Tebmar, A. Gopferich, European Journal of Pharmaceutics and Biopharmaceutics 2008, 68, 138) (shown in FIG. 1).

### Biological Imaging Using Water-Soluble Nanoparticle Imaging Agents

Generally, it is well known and accepted that cells regulate and control biological processes; but the mechanisms are poorly understood. A greater understanding of cells, the mechanisms by which cells function “normally”, and the deficiencies leading to disease states will provide a platform towards the “smart” design of therapeutics to correct cellular deficiencies to treat disease. One way to gain more insight into these mechanisms is cellular imaging of fixed and live cells using polymer-solubilized nanoparticles.

In addition to gaining a better understanding of cellular mechanisms, imaging techniques have been used to track nanomaterials within biological systems to determine where they accumulate as well as learn more about the relationship between nanomaterials and organisms (K. T. Thurn, E. M. B. Brown, A. Wu, S. Vogt, B. Lai, J. Maser, T. Paunesku, G. E. Woloschak, Nanoscale Research Letters 2007, 2, 430). One of the purposes of using a delivery vehicle to “carry” a therapeutic is to more effectively administer the therapeutic to the active site. Fluorescence imaging techniques can be used to instantaneously and terminally follow drug delivery in two ways: 1) organic fluorophores that do not affect the mechanism of the drug can be added to the vehicle or 2) nanoparticles (fluorescent or magnetic) can be formulated within the delivery vehicle and their release rates followed to model therapeutic release. In instance 1, the fate of the vehicle is followed, while in instance 2, the fate of a model therapeutic is profiled.

### Diagnostics

Fluorescence imaging techniques have also found widespread use in the diagnosis of disease, termed biosensing. A biosensor is built on two components; a biorecognition element and a signaling element. In the case of fluorescence-based biosensors, the signaling element, i.e. fluorescence, is often based upon Förster resonance energy transfer (FRET) in which energy is transferred from a donor chromophore to an acceptor chromophore resulting in a change in the fluorescence signal (enhancement or depletion). An example of a fluorescent biosensor is one in which the biorecognition element is an antibody that, upon binding with the protein or receptor it is specific to, produces a stronger fluorescent signal. The optimal biosensors are those that are selective to the desired target, fast, sensitive, produce accurate and reproducible results, and reusable.

### Theragnostics

Theragnostics are relatively new treatment techniques that combine disease diagnosis with a treatment modality. Theragnostics span a range of topics including predictive medicine, integrated medicine, and pharmocodiagnostics. The development and use of theragnostics has the potential to revolutionize disease therapy—shifting care towards “personalized medicine”. Polymer micelles are attractive candidates for development as theragnostics as they can carry multiple types of cargo.

### Nanoscale Amphiphilic Macromolecules

Nanoscale amphiphilic macromolecules (AMs) can form polymeric micelles. In aqueous media, the unimers self-assemble to form biocompatible, nano-sized micelles at concentrations as low at 100 nM (the CMC), making them more stable than other polymeric micellar systems that have CMCs only as low as 2 μM.

The basic unimer structure is a branched hydrophobic component formed by the tetra-alkylation of a biocompatible sugar, which is further derivatized with linear, hydrophilic PEG—all of which are linked via biodegradable bonds (L. Tian, L. Yam, N. Zhou, H. Tat, K. Ulrich, Macromolecules 2004, 37, 538). The synthesis of the parent compound, 1cM, from the biocompatible sugar, mucic acid, is shown in below.

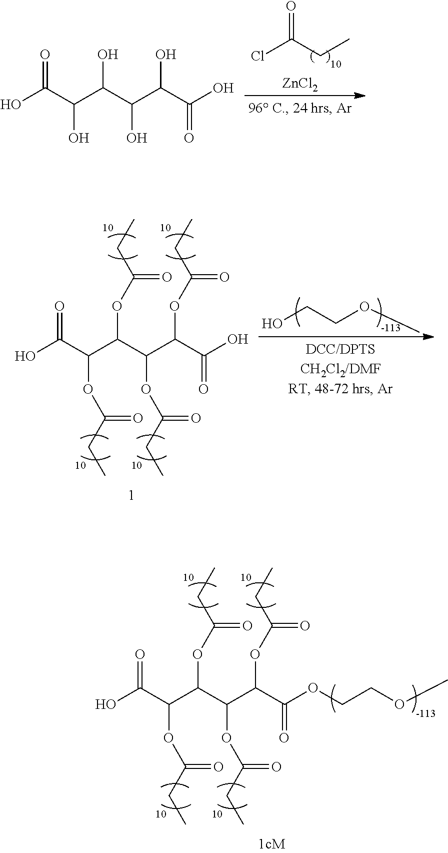

Many facets of the AM structure can be tuned depending upon the desired application: 1) hydrophobicity can be enhanced by the increasing the number of sugar hydroxyl groups (i.e., alkyl group modification points) as well as the increasing the alkyl group chain length used to derivatize the sugar, 2) hydrophilicity can be enhanced by lengthening the PEG length (or increasing molecular weight), and 3) sugar stereochemistry can be modified.

Sugar stereochemistry has been shown to be important in numerous biomedical applications. In work by Liu et. al. utilizing similar sugars incorporated into cationic polymers, the sugar stereochemistry had a significant affect on the complexation and delivery of siRNA (Y. Liu, T. M. Reineke, Journal of the American Chemical Society 2005, 127, 3004). In addition, it has been determined that the sugar stereochemistry has a significant effect on AM binding to scavenger receptors. While not intending to be a limitation, in this work, the sugar stereochemistry was held constant by utilizing a mucic acid backbone with the stereochemistry shown below.

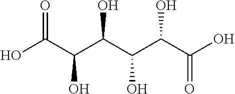

Previous research has employed AMs as polymer therapeutics for applications as drug delivery vehicles, to deliver cargo intra- and subcellularly, and as polymer drugs for the management of cardiovascular disease. However, AMs are attractive for multiple other applications due to their low CMC values, lending micellar stability, solution stability (lack of aggregation) for up to three weeks, and biocompatibility. As mentioned above, numerous applications-driven modifications can be made to tune AM hydrophobicity and hydrophilicity. Further, AM functional groups can also be modified on both the hydrophilic and hydrophobic portions of the unimer. The modification of the hydrophobic portion with functional groups for specific biomedical applications is discussed below.

Previous research has investigated 1cM as a biocompatible, micellar polymer therapeutic for the delivery of cancer therapeutics. By utilizing the free carboxylic acid within the hydrophobic moiety as a point of functionalization, the AMs can be modified in numerous ways to be used in a variety of biomedical applications. As discussed below, the ability of AMs to biostabilize white light-emitting nanocrystals was explored utilizing encapsulation and ligand exchange to design a novel imaging, biosensing, and/or theragnostic system.

### AMs to Biostabilize White Light-Emitting Nanocrystals

New safe and efficient fluorescence systems for imaging, biosensing, and other applications as discussed above are highly desirable for the design of better treatment options. Fluorescent nanocrystals are hydrophobic and often cytotoxic due to their heavy metal composition. To utilize fluorescent nanocrystals for biomedical imaging applications, the nanocrystals should be water-soluble. As discussed in the Examples below, white light-emitting nanocrystals (WLNCs) were biostabilized by encapsulation within AMs. Additionally, ligand exchange, whereby a functionalized AM coordinated to the metal surface of the nanocrystal, is also contemplated. The WLNCs were successfully water-solubilized using both an unmodified (1cM) and phosphonic acid-modified AM (1pM) without significantly hindering the nanocrystal fluorescence. The highest fluorescence was achieved with the AM-encapsulated WLNCs (1cM), while the smallest sized systems were achieved using ligand exchange-solubulized WLNC (1pM). Preliminary in vitro uptake in human THP-1 macrophage cells qualitatively showed no significant decrease in cell numbers or morphology change, indicating excellent cytocompatibility and more efficient uptake of the ligand exchange-solubilized WLNC.

By utilizing the carboxylic acid within the hydrophobic portion of the unimer, many new polymers have been designed and synthesized as discussed below. The Examples indicate that AMs are useful for the biostabilization of fluorescent nanocrystals for imaging, diagnostics, and/or biosensing applications.

### Semiconductor Nanocrystals

Semiconductor nanocrystals, better known as fluorescent nanocrystals or quantum dots (QDs), are extremely small (1-20 nm) nanoparticles composed of semiconductor materials such as CdSe, CdS, or PbSe. The utility of QDs is realized due to the electronic structure of the materials, which lies between that of single molecules and bulk semiconductors, due to quantum confinement. This property yields unique optical properties—e.g., broadband absorption and narrow (often single wavelength) emission based upon crystal size, shape, and composition. Thus, multiple particles of the same composition with only modifications to the crystal size can be excited at the same wavelength, while emitting at different, very specific wavelengths (R. J. Martin-Palma, M. Manso, V. Tones-Costa, Sensors 2009, 9, 5149).

In addition to having optical properties useful for multiplexed imaging experiments, QDs have numerous other advantages over organic fluorophores including superior photostability and fluorescence lifetimes, high quantum yields, and a high resistance to chemical degradation. In combination, these properties make QDs extremely versatile with respect to their applications. Thus, they have been employed in lighting applications, photovoltaic devices and biomedical applications discussed above, including tracking, imaging, diagnostics, theragnostics and biosensing (C. S. S. R. Kumar, Nanomaterials for Biosensors, Vol. 8, Wiley-VCH, Germany, 2007). They are particularly valuable in these biomedical applications as their size is comparable to that of many biomolecules, which is useful in biomolecule imaging and detection.

### Water-Solubilization of ODs

As mentioned above, as synthesized, the metal surface of QDs can be coated with hydrophobic alkanes such as tri-octyl phosphine oxide (TOPO), hexadecylamine, or octadecylamine which solubilize the metal precursors during synthesis. The alkanes also protect the resulting nanocrystals from surface modifications, such as oxidation and/or acid etching, that decrease or deactivate fluorescence properties of QDs. The deactivation of the optical properties results from the high atom surface-to-volume ratio that renders their surfaces extremely reactive; surface modification provides pathways for excited electrons to become “captured” and thus the emission of their energy through radiative processes is quenched. Overall, the presence of these ligands on the surface renders the QDs hydrophobic, which is problematic for biological applications.

In addition to QDs being hydrophobic, due to their heavy metal composition, QDs are not biocompatible. Thus, for use in biological applications, QDs should be modified to render them both hydrophilic and biocompatible.

Two methods that can be used to induce hydrophilicity and biocompatibility to QDs are encapsulation and ligand exchange (shown in FIG. 1).

Ideally, for biomedical applications, the method to induce water solubility, should yield systems that result in: 1) protection of the nanocrystal from surface reactions, 2) maintenance of small diameters, and 3) are amenable to further functionalization depending on the application, such as the ability to add targeting groups to enhance specificity.

### Encapsulation of QDs

Encapsulation can be used to biostabilize QDs in which the QDs as synthesized, complete with their organic ligands, are directly entrapped within amphiphilic systems such as polymer micelles or liposomes (C. S. S. R. Kumar, Nanomaterials for Biosensors, Vol. 8, Wiley-VCH, Germany, 2007). The driving force for the encapsulation is purely based upon hydrophobic interactions between the hydrophobic QDs and the hydrophobic component of the amphiphilic system (the lipid or the polymer). The advantage of encapsulation over other methods of solubilization is the ability to preserve the photophysical properties of the QDs because the surface of the crystals has not been altered. In contrast, encapsulation generally results in a significant increase in the size of the water-soluble QD systems.

### Ligand Exchange to Water-Solubilize ODs

A second method to biostabilize QDs is surface modification of the QDs with a water-soluble component via ligand exchange. This method of water-solubilization can be achieved using proteins or antibodies specific to the desired application or using organic acids, polymers or lipids that can directly interact with the metal surface of the QDs via coordination. The process of place-exchanging the hydrophobic ligands with water-soluble components on the surface of the quantum dots is called ligand exchange. This method of passivation results in a stronger interaction between the nonmetal of the coating and the nanocrystals (coordination of the nonmetal to the metal). This method of solubilization generally leads to sizes close to that of the original nanocrystal.

### White Light-Emitting Nanocrystals (WLNC)

“Magic sized” QDs are 1.5-1.7 nm nanocrystals that have broadband absorption and broadband fluorescence emission properties, producing white light upon excitation with UV light (M. J. Bowers II, J. R. McBride, S. J. Rosenthal, Journal of the American Chemical Society 2005, 127, 15378). As previously discussed, conventional QDs are broad-absorbing and narrow-emitting. This narrow emission is largely determined based upon band-edge emission, or the direct recombination of an electron and hole within the nanocrystal. However, another type of emission, termed deep-trap emission, occurs in CdSe QDs when photogenerated holes created on the nanocrystal surface due to non-coordinated selenium atoms encounter an excited electron before it can relax non-radiatively. In small nanocrystals (<3.0 nm), deep-trap emission is quite common since the surface-to-volume ratio is higher than larger QDs, meaning that uncoordinated selenium sites are available to trap electron holes. However, the presence of a large band-edge emission feature allows the QDs to maintain their narrow emission features. In WLNCs, a so-called “magic-size” was achieved such that the band-edge emission features are largely diminished. The result is that the optical emission spectra of the WLNCs is dominated by deep-trap emission, producing balanced white-light. Further, due to the short growth times necessary for the fabrication of WLNCs, their surfaces are likely to have more defects than conventional QDs and a larger ratio of uncoordinated surface selenium atoms.

These WLNCs have been investigated for their utility as white-LEDs, because they do not suffer from self-absorption like other QD systems used to produce white-light. Beyond solid-state lighting, the unique efficiency of their white-light emission has the potential for biomedical applications, particularly for biosensing applications where white light-emission could be used to simultaneously sense multiple analytes.

As discussed above, to utilize QDs in biological applications, the systems should first be water-solubilized. In this respect, WLNCs are no different than traditional QDs. However, due mostly to their small size, their surface-to-volume ratio is even larger than typical QDs. While they may have surface reactivity equivalent to that of other CdSe QDs, surface modifications have a greater effect on the overall photochemical properties, making WLNCs particularly sensitive to their environment. However, harnessed correctly, this environmental sensitivity could be the key to utilizing WLNCs as biosensors.

### Certain Embodiments of Amphiphilic Macromolecules and Encapsulates

As described herein, amphiphilic macromolecules are used to encapsulate nanocrystals. Certain amphiphilic macromolecules include those described in the Examples. Certain embodiments of the invention are directed to these amphiphilic macromolecules.

In one embodiment an amphiphilic macromolecule of formula (I):

D-X—Y—Z—R₁ (I)

wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; R₁ is a polyether; R^{a} is a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alkyl.

In one embodiment R^{a} is a (C₁-C₁₀)alkyl group substituted with one or more groups independently selected from —COOH, —P(═O)(OH)₂, —OP(═O)(OH)₂, —S(═O)₂(OH), —OSO₃H, and —ON(═O)OH, and optionally substituted with one or more —COOH, or sulfo S(═O)₂OH groups.

In one embodiment, R^{a} is a (C₁-C₁₀)alkyl group substituted with one or more groups independently selected from —OH, —N(R^{b})₂, —SH, and —P(R^{c})₂, and optionally substituted with one or more —COOH, or sulfo S(═O)₂OH groups.

In one embodiment an amphiphilic macromolecule is a compound of formula (I):

D-X—Y—Z—R₁ (I)

wherein D is —C(═O)NR^{a}R^{b}; X is a polyol, Y is —C(═O)—, —C(═S)—, or is absent; Z is O, S or NH; R₁ is a polyether; R^{a} is a (C₁-C₁₀)alkyl group substituted with one or more —P(═O)(OH)₂ groups and optionally substituted with one or more carboxy COOH, or sulfo S(═O)₂OH groups; and R^{b} is H or (C₁-C₆)alkyl; wherein one or more hydroxy groups of the polyol are acylated with a fatty acid residue.

In one embodiment R^{a} is a (C₁-C₁₀)alkyl group substituted with one or two —P(═O)(OH)₂ groups and optionally substituted with one or more carboxy COOH, or sulfo S(═O)₂OH groups.

In one embodiment R^{a} is a (C₁-C₁₀)alkyl group substituted with one —P(═O)(OH)₂ group and optionally substituted with one or more carboxy COOH, or sulfo S(═O)₂OH groups.

In one embodiment R^{a} is a (C₁-C₆)alkyl group substituted with one or two —P(═O)(OH)₂ groups and optionally substituted with one or more carboxy COOH, or sulfo S(═O)₂OH groups.

In one embodiment R^{a} is a (C₁-C₆)alkyl group substituted with one —P(═O)(OH)₂ group and optionally substituted with one or more carboxy COOH, or sulfo S(═O)₂OH groups.

In one embodiment R^{a} is a (C₁-C₆)alkyl group substituted with one or two —P(═O)(OH)₂ groups and optionally substituted with one carboxy COOH group.

In one embodiment R^{a} is a (C₁-C₆)alkyl group substituted with one —P(═O)(OH)₂ group and optionally substituted with one sulfo S(═O)₂OH group.

In one embodiment R^{a} is a (C₃-C₆)alkyl group substituted with one or two —P(═O)(OH)₂ groups and optionally substituted with one or more carboxy COOH, or sulfo S(═O)₂OH groups.

In one embodiment R^{a} is a (C₃-C₆)alkyl group substituted with one —P(═O)(OH)₂ group and optionally substituted with one or more carboxy COOH, or sulfo S(═O)₂OH groups.

In one embodiment R^{a} is a (C₃-C₆)alkyl group substituted with one or two —P(═O)(OH)₂ groups and optionally substituted with one carboxy COOH group.

In one embodiment R^{a} is a (C₃-C₆)alkyl group substituted with one —P(═O)(OH)₂ group and optionally substituted with one sulfo S(═O)₂OH group.

In one embodiment R^{a} is a propyl group substituted with one or two —P(═O)(OH)₂ groups and optionally substituted with one or more carboxy COOH, or sulfo S(═O)₂OH groups.

In one embodiment R^{a} is a propyl group substituted with one —P(═O)(OH)₂ group and optionally substituted with one or more carboxy COOH, or sulfo S(═O)₂OH groups.

In one embodiment R^{a} is a propyl group substituted with one or two —P(═O)(OH)₂ groups and optionally substituted with one carboxy COOH group.

In one embodiment R^{a} is a propyl group substituted with one —P(═O)(OH)₂ group and optionally substituted with one sulfo S(═O)₂OH group.

In one embodiment R^{a} is a (C₃-C₆)alkyl group substituted with one —P(═O)(OH)₂ group and one carboxy COOH group.

In one embodiment R^{a} is a propyl group substituted with one —P(═O)(OH)₂ group and one carboxy COOH group.

In one embodiment R^{a} is a (C₃-C₆)alkyl group substituted with one —P(═O)(OH)₂ group.

In one embodiment R^{a} is a propyl group substituted with one —P(═O)(OH)₂ group.

In one embodiment the polyol has from 2 carbons to 20 carbons.

In one embodiment the polyol has from 3 carbons to 12 carbons.

In one embodiment the polyol has from 4 carbons to 10 carbons.

In one embodiment the polyol comprises from 2 to 20 hydroxy groups.

In one embodiment the polyol comprises from 2 to 12 hydroxy groups.

In one embodiment the polyol comprises from 2 to 10 hydroxy groups.

In one embodiment the polyol is substituted with one or more carboxy groups.

In one embodiment the polyol is substituted with two carboxy groups.

In one embodiment the polyol is substituted with one carboxy group.

In one embodiment the polyol is a mono- or di-carboxyllic acid containing from 1 to about 10 carbon atoms and substituted with from 1 to about 10 hydroxyl groups.

In one embodiment the polyol is mucic acid, malic acid, citromalic acid, alkylmalic acid, hydroxy derivatives of glutaric acid, alkyl glutaric acids, tartaric acid, or citric acid.

In one embodiment the polyol is mucic acid.

In one embodiment the polyol is 2,2-(bis(hydroxymethyl)propionic acid, or tricine.

In one embodiment the polyol is a saccharide.

In one embodiment the polyether is a poly(alkylene oxide) having between about 2 and about 150 repeating units.

In one embodiment the alkylene oxide units contain from 2 to 4 carbon atoms and may be straight chained or branched.

In one embodiment the polyether is alkoxy-terminated.

In one embodiment the polyether is linked to the polyol through an ester, thioester, or amide linkage.

In one embodiment the polyether is linked to the polyol through an ester or amide linkage.

In one embodiment the polyether has the following structure:

R₅—(R₆—O—)ₐ—R₆-Q-

wherein R₅ is a 1 to 20 carbon straight-chain or branched alkyl group, —OH, —OR₇, —NH₂, —NHR₇, —NHR₇R₈, —CO₂H, —SO₃H (sulfo), —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(═O)—O—; R₆ is a 1 to 10 carbon straight-chain or branched divalent alkylene group; each R₇ and R₈ is independently a 1 to 6 carbon straight-chain or branched alkylene group; Q is —O—, —S—, or —NR₇; and a is an integer from 2 to 110, inclusive.

In one embodiment the polyether is a methoxy terminated polyethylene glycol.

In one embodiment the fatty acids comprise from 2 to 24 carbon atoms.

In one embodiment the fatty acids comprise from 6 to 18 carbon atoms.

In one embodiment each fatty acid comprises 12 carbon atoms.

In one embodiment the fatty acids comprise caprylic, capric, lauric, myristic, myristoleic, palmitic, palmitoleic, stearic, oleic, linoleic, arachidic, behenic, or erucic acid, or a mixture thereof.

In one embodiment Y is —C(═O)—; Z is O; and R₁ is —(CH₂CH₂O)₁₁₃—CH₃.

Certain embodiments provide a macromolecule of formula (I), wherein D is:

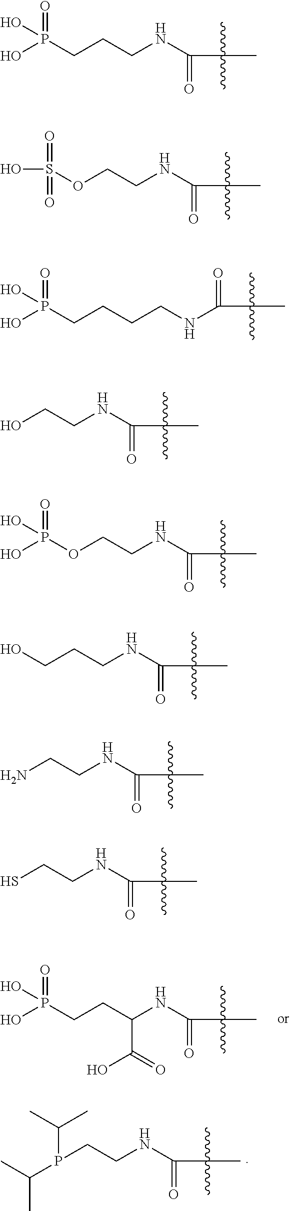

In one embodiment the macromolecule as described herein is:

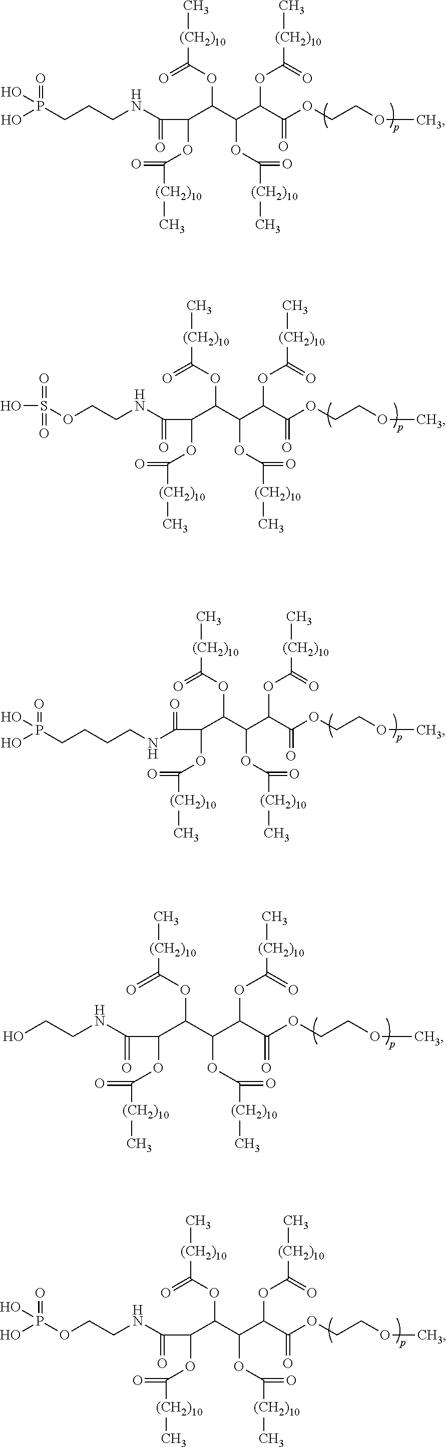

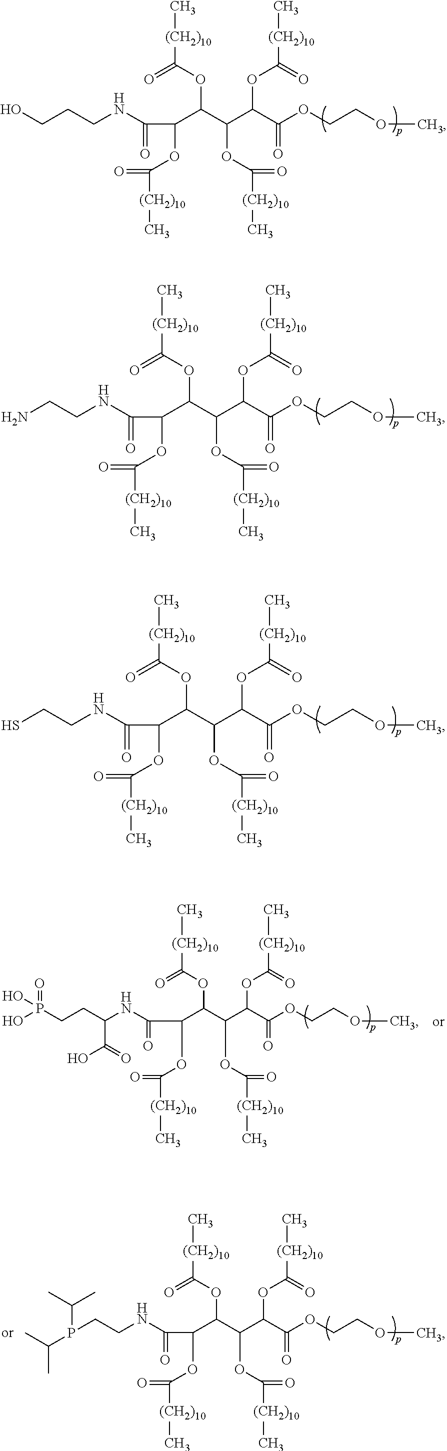

wherein p is an integer in the range from about 90 to about 140.

In one embodiment p is an integer in the range from about 105 to about 125.

In one embodiment p is an integer in the range from about 110 to about 120.

In one embodiment p is an integer in the range from about 90 to about 140.

In one embodiment p is about 110, 111, 112, 113, 114, 115, or 116.

In one embodiment p is about 113.

Accordingly, certain embodiments provide a compound of formula (I) as described above. Such compounds of formula (I) are useful for preparing encapsulates as described herein.

Certain embodiments also provide a composition comprising a plurality of compounds of formula (I) in a solvent. Such a composition is useful for preparing encapsulates as described herein.

Certain embodiments provide a composition comprising a plurality of macromolecules as described herein (e.g. of formula (I)) in a solvent.

Certain embodiments also provide a composition comprising a plurality of compounds of formula (I) in a solvent, wherein the compounds of formula (I) are associated into one or more aggregates.

Certain embodiments provide a composition comprising a plurality of macromolecules as described herein (e.g. of formula (I)), in a solvent, wherein the macromolecules form one or more aggregate structures.

Compounds of formula (I) having unsaturated bonds (e.g., in the fatty acid or polyether groups), can be cross-linked after aggregate formation to form covalently bound structures (i.e. cross-linked micelles).

Accordingly, certain embodiments also provide a composition comprising a cross-linked micelle that is formed from plurality of compounds of formula (I) in a solvent, wherein the compounds of formula (I) form one or more aggregate structures and have been cross-linked to provide the cross-linked micelle.

Certain embodiments provide a composition comprising a solvent, and a cross-linked micelle comprising a plurality of macromolecules as described herein (e.g. of formula (I)), wherein the macromolecules form one or more aggregate structures that are cross-linked to provide the cross-linked micelle.

Certain embodiments provide an aggregate structure formed by combining a plurality of macromolecules as described herein (e.g. of formula (I)), in a solvent and allowing the macromolecules to form the aggregate.

Certain embodiments provide a cross-linked micelle formed by combining a plurality of macromolecules as described herein (e.g. of formula (I)), in a solvent, allowing the macromolecules to form aggregate structures, and cross-linking the macromolecules to provide the cross-linked micelle.

Certain embodiments also provide a method for preparing a cross-linked micelle as described herein comprising cross-linking aggregates comprising a plurality of compounds of formula (I) to provide the cross-linked micelle. Certain embodiments further provide a method where the aggregates are formed by combining a plurality of compounds of formula (I) in a solvent.

Certain embodiments provide an encapsulate comprising a molecule surrounded or partially surrounded by a plurality of macromolecules as described herein (e.g. of formula (I)).

Certain embodiments provide an encapsulate comprising a molecule surrounded or partially surrounded by a plurality of macromolecules of formula (I) or residues thereof.

Certain embodiments also provide a composition comprising a solvent, and an aggregate of a plurality of compounds of formula (I) surrounding a nanocrystal.

Certain embodiments provide an encapsulate comprising a nanocrystal surrounded or partially surrounded by a plurality of macromolecules as described herein (e.g. of formula (I)).

Certain embodiments provide an encapsulate comprising a nanocrystal surrounded or partially surrounded by a plurality of macromolecules of formula (I):

D-X—Y—Z—R₁ (I)

or residues thereof, wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; each R₁ is independently a polyether; each R^{a} is independently a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alkyl.

Certain embodiments provide an encapsulate comprising a nanocrystal in a cross-linked micelle, which cross-linked micelle comprises a plurality of macromolecules as described herein (e.g. of formula (I)), which have been cross-linked.

Certain embodiments provide an encapsulate comprising a nanocrystal surrounded or partially surrounded by an aggregate or a cross-linked micelle as described herein.

Certain embodiments provide an encapsulate formed by combining a plurality of macromolecules as described herein (e.g. of formula (I)) and a nanocrystal, in a solvent; and allowing the macromolecules to encapsulate the nanocrystal.

Certain embodiments provide an encapsulate formed by combining a) a plurality of macromolecules of formula (I):

D-X—Y—Z—R₁ (I)

wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; each R₁ is independently a polyether; each R^{a} is independently a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alky; b) a nanocrystal, and c) a solvent; and allowing the macromolecules and nanocrystals to form the encapsulate.

Certain embodiments provide a method for preparing an encapsulate as described herein comprising combining a plurality of compounds of formula (I) and a nanocrystal in a solvent, and allowing the compounds of formula (I) to aggregate around the nanocrystal, to provide the encapsulate (i.e. the nanocrystal surrounded or partially surrounded by a plurality of compounds of formula (I)).

Certain embodiments also provide a method for preparing an encapsulate as described herein comprising combining a plurality of compounds of formula (I) and a nanocrystal in a solvent, allowing the compounds of formula (I) to aggregate around the molecule, and cross-linking the compounds of formula (I) to provide the encapsulate (i.e. the nanocrystal in a cross-linked micelle).

Certain embodiments provide the nanocrystal is a light-emitting nanocrystal.

Certain embodiments provide the nanocrystal is a white-light-emitting nanocrystal.

Certain embodiments provide the nanocrystal is a quantum dot.

Certain embodiments provide a pharmaceutical composition comprising an encapsulate as described herein and a pharmaceutically acceptable carrier.

Certain embodiments provide a method for delivering a nanoparticle to an animal comprising administering an encapsulate as described herein to the animal.

Certain embodiments provide a method for delivering a nanocrystal to an animal comprising administering the composition as described herein to the animal.

Certain embodiments provide an encapsulate as described herein for use in medical treatment or diagnosis.

Certain embodiments provide an encapsulate as described herein for use in therapy.

Certain embodiments provide intermediates and processes useful for preparing compounds of formula (I) as described herein.

As used herein the term “polyol” includes straight chain and branched chain aliphatic groups, as well as mono-cyclic and poly-cyclic aliphatics, which are substituted with two or more hydroxy groups. A polyol typically has from about 2 carbons to about 20 carbons; preferably, from about 3 carbons to about 12 carbons; and more preferably from about 4 carbons to about 10 carbons. A polyol also typically comprises from about 2 to about 20 hydroxy groups; preferably from about 2 to about 12 hydroxy groups; and more preferably from about 2 to about 10 hydroxy groups. A polyol can also optionally be substituted on a carbon atom with one or more (e.g., 1, 2, or 3) carboxy groups (COOH). These carboxy groups can conveniently be used to link the polyol to the polyether in a compound of formula (I).

One specific polyol is a mono- or di-carboxyllic acid containing from 1 to about 10 carbon atoms and substituted with from 1 to about 10 hydroxyl groups. The mono- or di-carboxylic acid may be a straight chained or branched chained aliphatic, or a mono-cyclic or poly-cyclic aliphatic compound. Suitable dicarboxylic acids include mucic acid, malic acid, citromalic acid, alkylmalic acid, hydroxy derivatives of glutaric acid, and alkyl glutaric acids, tartaric acid, citric acid, hydroxy derivatives of rumadic acid, and the like. Suitable monocarboxylic acids include 2,2-(bis(hydroxymethyl)propionic acid, and N-[tris(hydroxymethyl)methyl]glycine (tricine).

Another specific polyol is a “saccharide,” which includes monosaccharides, disaccharides, trisaccharides, polysaccharides and sugar alcohols. The term includes glucose, sucrose fructose and ribose, as well as deoxy sugars such as deoxyribose and the like. Saccharide derivatives can conveniently be prepared by methods known to the art. Examples of suitable mono-saccharides are xylose, arabinose, and ribose. Examples of di-saccharides are maltose, lactose, and sucrose. Examples of suitable sugar-alcohols are erythritol and sorbitol.

As used herein, the term polyether includes poly(alkylene oxides) having between about 2 and about 150 repeating units. Typically, the poly(alkylene oxides) have between about 50 and about 110 repeating units. The alkylene oxide units contain from 2 to 10 carbon atoms and may be straight chained or branched. Preferably, the alkylene oxide units contain from 2 to 10 carbon atoms. Poly(ethylene glycol) (PEG) is preferred. Alkoxy-, amino-, carboxy-, and sulfo-terminated poly(alkylene oxides) are preferred, with methoxy-terminated poly(alkylene oxides) being more preferred.

A preferred polyether has the following structure:

R₅—(R₆—O—)ₐ—R₆-Q-

wherein R₅ is a 1 to 20 carbon straight-chain or branched alkyl group, —OH, —OR₇, —NH₂, —NHR₇, —NHR₇R₈, —CO₂H, —SO₃H (sulfo), —CH₂—OH, —CH₂—R₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(˜O)—O—;

R₆ is a 1 to 10 carbon straight-chain or branched divalent alkylene group;

each R₇ and R₈ is independently a 1 to 6 carbon straight-chain or branched alkylene group;

Q is —O—, —S—, or —NR₇; and

a is an integer from 2 to 150, inclusive.

Another preferred polyether is methoxy terminated polyethylene glycol.

In a compound of formula (I), a poly(alkylene oxide) can be linked to a polyol, for example, through an ether, thioether, amine, ester, thioester, thioamide, or amide linkage. Preferably, a poly(alkylene oxide) is linked to a polyol by an ester or amide linkage in a compound of formula (I).

As used herein, the term fatty acid includes fatty acids and fatty oils as conventionally defined, for example, long-chain aliphatic acids that are found in natural fats and oils. Fatty acids typically comprise from about 2 to about 24 carbon atoms. Preferably, fatty acids comprise from about 6 to about 18 carbon atoms. The term “fatty acid” encompasses compounds possessing a straight or branched aliphatic chain and an acid group, such as a carboxylate, sulfonate, phosphate, phosphonate, and the like. The “fatty acid” compounds are capable of “esterifying” or forming a similar chemical linkage with hydroxy groups on the polyol. Examples of suitable fatty acids include caprylic, capric, lauric, myristic, myristoleic, palmitic, palmitoleic, stearic, oleic, linoleic, eleostearic, arachidic, behenic, erucic, and like acids. Fatty acids can be derived from suitable naturally occurring or synthetic fatty acids or oils, can be saturated or unsaturated, and can optionally include positional or geometric isomers. Many fatty acids or oils are commercially available or can be readily prepared or isolated using procedures known to those skilled in the art.

As used herein, the term “aggregate” means a plurality of compounds of formula (I) in a solvent that have organized into an ordered structure, for example, a structure having a hydrophobic core and a surrounding hydrophilic layer, or a structure having a hydrophilic core and a surrounding hydrophobic layer.

As used herein, the term “a plurality of compounds of formula (I)” means more than one compound of formula (I). In such a plurality, each compound of formula (I) can have the same structure, or the plurality can include compounds of formula (I) that have differing structures. In a preferred embodiment, the term “a plurality of compounds of formula (I)” means more than one compound of formula (I), wherein each of the compounds of formula (I) has the same structure.

As used herein, the term functionalized amphiphilic macromolecule (AM) (e.g. compounds of formula (I)) means a functional group has been chemically incorporated within the hydrophobic portion of the polymer backbone of an amphiphilic macromolecule. For example, functional groups may include acidic groups, such as sulphate and phosphate, basic moieties, such as amines and hydroxyls, and soft ligands, such as organophosphines and thiols.

As used herein, a “cross-linked micelle” means an aggregate that has been cross-linked to provide a covalently cross-linked structure.

As described herein, amphiphilic macromolecules are used to encapsulate nanocrystals. In certain embodiments, the nanocrystals are fluorescent nanocrystals. In certain embodiments, the nanocrystals are white-light emitting nanocrystals. In certain embodiments, the nanocrystals are quantum dots. Certain nanocrystals are described, e.g., in Bowers et al., Journal of the American Chemical Society, 131 (16): 5730-5731, including supporting information (2009) and in M. J. Bowers II, J. R. McBride, S. J. Rosenthal, Journal of the American Chemical Society 2005, 127, 15378.

As used herein the term “macromolecules of formula (I) or residues thereof” encompass macromolecules having formula (I) as well as portions of the macromolecules of formula (I) that encapsulate a molecule or nanocrystal after undergoing, for example, ligand exchange with the surface of the molecule or nanocrystal.

As used herein, the term “encapsulate” means an aggregate, having a nanocrystal surrounded or partially surrounded by a plurality of compounds of formula (I). The term encapsulate includes structures wherein the compounds of formula (I) have been cross-linked, as well as structures wherein the compounds of formula (I) have not been cross-linked. The term “encapsulate” also includes structures wherein the compounds of formula (I) have undergone ligand exchange with the surface of the nanocrystal or wherein the compounds of formula (I) ligand cap the nanocrystal (i.e. encapsulation). Encapsulate may also include compounds of formula (I) surrounding or partially surrounding a nanocrystal, which has ligands on its surface (e.g. non-AM ligands, such as TOPO and octadecylphosphonic acid).

As used herein, the term “stabilized encapsulate” means an aggregate, having a nanocrystal surrounded or partially surrounded by a plurality of compounds of formula (I), wherein unsaturated bonds in the compounds of formula (I) have been cross-linked to provide a covalently stabilized structure.

When a plurality of compounds of formula (I) are placed in a hydrophilic solvent (e.g., an aqueous solution comprising water), Applicant has discovered that the compounds of formula (I) will aggregate, with the polyether portion of the compounds extending into the hydrophilic solvent, and the fatty acid portions of the compounds forming a hydrophobic core. Such aggregates can solubilize a nanocrystal, by encapsulating the nanocrystal in the fatty acid core of the aggregates and/or ligand exchange on the surface of the nanocrystal. The nanocrystal can typically be added to the solution of the compounds of formula (I) subsequent to aggregation, or the hydrophobic molecule can be added to the solution of the compounds of formula (I) prior to aggregation, allowing the aggregates to form around the molecule. Thus, the aggregates formed from the compounds of formula (I) can function similar to traditional micelles.

As used herein, the term “surround” means the entire surface of the molecule or nanocrystal is coated or encompassed by one or more compounds of formula (I).

As used herein, the term “partially surround” means at least a portion of the surface of the molecule or nanocrystal is coated or encompassed by one or more compounds of formula (I) and a portion of the surface of the molecule or nanocrystal is not coated or encompassed (e.g. is exposed).

The encapsulates as described herein can be formulated as pharmaceutical compositions and administered to a mammalian host, such as a human patient in a variety of forms adapted to the chosen route of administration.

The encapsulates as described herein may be administered intravenously or intraperitoneally by infusion or injection. Solutions of the encapsulates can be prepared, for example, in water. Under ordinary conditions of storage and use, these preparations may contain a preservative to prevent the growth of microorganisms.

The pharmaceutical dosage forms suitable for injection or infusion should be sterile, fluid and stable under the conditions of manufacture and storage. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the encapsulates as described herein in the required amount in the appropriate solvent with various of the other ingredients enumerated above, as required, followed by sterilization.

The dose and method of administration will vary from animal to animal and be dependent upon such factors as the type of animal being treated, its sex, weight, diet, concurrent medication, overall clinical condition, the particular therapeutic agent employed, the specific use for which the agent is employed, and other factors which those skilled in the relevant field will recognize.

Therapeutically effective dosages may be determined by either in vitro or in vivo methods. For each particular dosage form, individual determinations may be made to determine the optimal dosage required. The range of therapeutically effective dosages will naturally be influenced by the route of administration, the therapeutic objectives, and the condition of the patient. The determination of effective dosage levels, that is, the dosage levels necessary to achieve the desired result, will be within the ambit of one skilled in the art. Typically, applications of agent are commenced at lower dosage levels, with dosage levels being increased until the desired effect is achieved.

Certain embodiments of the invention will now be illustrated by the following non-limiting Examples.

### Example 1

As shown below, AMs can be used to biostabilize WLNC due to their ability to encapsulate hydrophobic materials and the ability to modify the AMs for ligand exchange. By achieving a balance between surface interaction and encapsulation, the WLNC can be water-solubilized without affecting their emission properties while maintaining a nanoscale size.

In this Example, it is shown that AMs can be used to water-solubilize the WLNCs while maintaining the fluorescence emission properties and nanoscale sizes of the unmodified WLNCs. For water-solubilization, methods of encapsulation and ligand exchange were explored utilizing two AMs; a carboxy-terminated AM and a functionalized AM capable of coordinating to the nanocrystal surface. The properties of the water-solubilized WLNCs were evaluated via fluorescence spectroscopy and dynamic light scattering (DLS) with the goal of maintaining the white-light fluorescence emission and small, nanoscale size with the water-solubilized WLNCs. The highest fluorescence was achieved with AM-encapsulated WLNCs while the smallest sized systems were achieved using ligand exchange-solubulized WLNC. However, the functionalized polymer capable of coordinating to the nanocrystal surface produced the optimal balance of fluorescence intensity and nanoscale size desired. Additionally, in vitro uptake in human THP-1 macrophage cells qualitatively showed no significant decrease in cell numbers or morphology change, indicating excellent cytocompatibility, and more efficient uptake of the ligand exchange-solubilized WLNC. In general, these data highlight the ability to utilize AMs and functionalized AMs as a coating for insoluble, cytotoxic fluorescent nanocrystals without altering their emission properties. Additionally, this Example demonstrates that AM-solubilized WLNCs may prove to be highly useful for future biological applications, specifically in biosensing applications.

### Materials and Methods

Synthetic Materials. Unless otherwise stated, solvents and reagents were purchased from Fisher Scientific (Pittsburgh, Pa.) and Sigma-Aldrich (St. Louis, Mo.) and used as received. PEG 5 kDa was purchased from Polysciences, Inc. (Warrington, Pa.) and dried by azeotropic distillation from toluene before use. The 1cM and 0cM (starting material for 1pM) were synthesized as previously described (L. Tian, L. Yam, N. Zhou, H. Tat, K. Uhrich, Macromolecules 2004, 37, 538; J. Djordjevic, L. Del Rosario, J. Wang, K. Uhrich, Journal of Bioactive and Compatible Polymers 2008, 23, 532) and the CdSe WLNCs were synthesized and provided as solutions in chloroform by Professor Sandra Rosenthal's group at Vanderbilt University, Department of Chemistry (M. J. Bowers II, J. R. McBride, S. J. Rosenthal, Journal of the American Chemical Society 2005, 127, 15378).

Proton Nuclear Magnetic Resonance (¹H NMR) Spectroscopy. Proton nuclear magnetic resonance (¹H-NMR) spectra of the products were obtained using a Varian 400 MHz or 500 MHz spectrophotometer. Samples were dissolved in chloroform-d, with a few drops of dimethyl sulfoxide-d₆ if necessary, with tetramethylsilane as an internal reference.

Gel Permeation Chromatography (GPC). Molecular weights (M_{w}) and polydispersity indices (PDI) were determined using gel permeation chromatography (GPC) with respect to PEG standards (Sigma-Aldrich) on a Waters Stryagel® HR 3 THF column (7.8×300 mm). The Waters LC system (Milford, Mass.) was equipped with a 2414 refractive index detector, a 1515 isocratic HPLC pump, and 717plus autosampler. An IBM ThinkCentre computer with Waters Breeze Version 3.30 software installed was used for collection and processing of data. Samples were prepared at a concentration of 10 mg/mL in tetrahydrofuran, filtered using 0.45 gm pore size nylon or poly(tetrafluoroethylene) syringe filters (Fisher Scientific) and placed in sample vials to be injected into the system.

1pM Polymer Synthesis. In a 100 mL round bottom flask, 3-aminopropyl phosphonic acid (110 mg, 0.79 mmol) was dissolved in HPLC-grade H₂O (6 mL), HPLC-grade THF (12 mL), and triethylamine (0.38 mL, 2.7 mmol) and the solution stirred at room temperature. In a separate flask, 0cM (1.10 g, 0.183 mmol) was dissolved in HPLC-grade THF (25 mL) and the solution added to the reaction flask. The yellow solution was stirred for 18-20 hrs at room temperature before the THF was removed by rotary evaporation. The resulting yellow oil was dissolved in CH₂Cl₂ and 0.1 N HCl and stirred for 20-30 min. The mixture was then transferred to a separatory funnel containing addition 0.1 N HCl and the organic layer separated and washed with brine (2×). The combined aqueous portions were extracted with CH₂Cl₂ and the combined organics dried over MgSO₄ and concentrated to a yellow oil. White product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with ether (1×). The white solid was dried under ambient conditions (48 hrs) and under high vacuum (12 hrs). Yield: 0.896 g, 80%. ¹H-NMR (CDCl₃): δ 5.70 (m, 2H, CH), 5.00 (m, 2H, CH), 4.20 (m, 4H, CH₂), 3.60 (m, ˜0.45 kH, CH₂O), 3.38 (s, 3H, CH₃), 2.43 (m, 8H, CH₂), 1.61 (m, 10H, CH₂), 1.22 (m, 64H, CH₂), 0.87 (t, 12H, CH₃). GPC: Mw: 7.0 kDa; PDI: 1.06.

Water-solubilization of WLNCs by Solvent Evaporation. Two samples each of WLNCs solubilized by each polymer were prepared by solvent evaporation. Exact amounts of all reagents and concentrations are specified in Table 1 below. For a polymer:WLNC ratio of 20:1, 150 μL of a 1.0 mM solution of WLNCs in chloroform (concentration determined as previously described in W. W. Yu, L. Qu, W. Guo, X. Peng, Chemistry of Materials 2003, 15, 2854) were added to a 1.25 mM solution of each polymer in chloroform (final volume—2.900 mL chloroform). The solutions were agitated on a shaker for 3 hours at room temperature before the chloroform was removed by rotary evaporation. The resulting yellow films were dried under flowing argon gas for one minute and under ambient conditions for at least 12 hours. Water-solubilization was achieved by adding water (5.000 mL) to achieve a concentration of ˜30.0 μM WLNCs and ˜600 μM polymer. Filtered samples were prepared by passing the solutions through 0.45 μm Fisherbrand nylon syringe filters (Fisher Scientific).

TABLE 1
Specific amounts and concentrations of polymers and WLNCs
used to formulate the water-solubilized nanocrystals.

| | | | | Stirring | | Water-Soluble WLNCs | | |
|---|---|---|---|---|---|---|---|---|
| | AM | AM | WLNCs | [AM] | [WLNC] | [AM] | [WLNC] | |
| Sample | (mg) | (μmol) | (μmol) | (μM) | (μM) | (μM) | (μM) | AM:WLNC |
| 1pM 1 | 20.9 | 3.43 | 0.150 | 1180 | 51.7 | 685.2 | 30.0 | 22.8 |
| 1pM 2 | 20.8 | 3.41 | 0.150 | 1180 | 51.7 | 682.0 | 30.0 | 22.7 |
| 1cM 1 | 19.4 | 3.29 | 0.150 | 1130 | 51.7 | 657.6 | 30.0 | 21.9 |
| 1cM 2 | 21.3 | 3.61 | 0.150 | 1240 | 51.7 | 722.0 | 30.0 | 24.1 |

Polymer-solubilized nanocrystal solutions with varying polymer:WLNC ratios were prepared using the general procedure described above. The specific amounts for each ratio are shown in Table 2, below. Generally, the appropriate amount of polymer necessary to obtain the desired polymer:WLNC ratio was dissolved in chloroform and 348 μL of a 0.935 mM solution of WLNCs in chloroform (concentration determined as previously described in W. W. Yu, L. Qu, W. Guo, X. Peng, Chemistry of Materials 2003, 15, 2854) were added (final volume—3.044 mL chloroform). The solutions were agitated on a shaker for 3 hours at room temperature before the chloroform was removed by rotary evaporation. The resulting yellow films were dried under flowing argon gas for one minute. Water-solubilization was achieved by adding water (15.000 mL) to achieve a concentration of 21.7 μM WLNCs.

TABLE 2
Specific amounts and concentrations of polymers and WLNCs used to formulate
the water-solubilized nanocrystals of varying polymer:WLNC ratios.

| | | | | Stirring | | Water-Soluble WLNCs | | |
|---|---|---|---|---|---|---|---|---|
| | AM | AM | WLNCs | [AM] | [WLNC] | [AM] | [WLNC] | |
| Sample | (mg) | (μmol) | (μmol) | (μM) | (μM) | (μM) | (μM) | AM:WLNC |
| 1pM 10 | 21.0 | 3.44 | 0.325 | 1130 | 107 | 230 | 21.7 | 10.6 |
| 1pM 20 | 40.1 | 6.57 | 0.325 | 2160 | 107 | 438 | 21.7 | 20.2 |
| 1pM 40 | 81.9 | 13.4 | 0.325 | 4140 | 107 | 895 | 21.7 | 41.3 |
| 1pM 60 | 120.1 | 19.7 | 0.325 | 6470 | 107 | 1313 | 21.7 | 60.5 |
| 1pM 80 | 158.2 | 25.9 | 0.325 | 8520 | 107 | 1730 | 21.7 | 79.7 |
| 1cM 10 | 21.4 | 3.57 | 0.325 | 1170 | 107 | 238 | 21.7 | 11.0 |
| 1cM 40 | 82.4 | 13.7 | 0.325 | 4510 | 107 | 916 | 21.7 | 42.2 |
| 1cM 80 | 164.6 | 27.4 | 0.325 | 9010 | 107 | 1830 | 21.7 | 84.3 |

Qualitative Evaluation of Turbidity and Fluorescence. Turbidity of filtered and unfiltered solutions of water-solubilized WLNCs as compared with WLNCs in chloroform at the same concentration was qualitatively evaluated by visual inspection and images captured using a Canon PowerShot SD400 digital camera. Fluorescence of the solutions was qualitatively evaluated following excitation with a Spectroline® Longlife™ Filter long wavelength UV lamp (365 nm) and images captured using a Canon PowerShot SD400 digital camera.

Quantitative Turbidity Percent Transmittance. Turbidity of filtered and unfiltered solutions of water-solubilized WLNCs as compared with WLNCs in chloroform at the same concentration was evaluated quantitatively by UVNisible absorbance on a Lambda Bio XLS instrument (Perkin Elmer, Waltham, Mass.) scanning from 190-650 nm.

Quantitative Fluorescence Emission. Fluorescence of water-solubilized WLNCs and WLNCs in chloroform at the same concentration from 380-800 nm was quantified using a Shimadzu RF-5301 PC spectrofluorophotometer, with an excitation wavelength of 365 nm. For simplicity, during data analysis duplicate samples were average and graphed.

Hydrodynamic Diameter. Hydrodynamic diameters were evaluated by dynamic light scattering (DLS) using a Malvern Instruments Zetasizer Nano ZS-90 instrument (Southboro, Mass.). DLS measurements were performed at a 90° scattering angle at 25° C. Size distributions by volume of measurements were collected in triplicate, averaged and reported.

Cellular Uptake in THP-1 Macrophages. Internalization of AM-solubilized WLNCs by THP-1 macrophage cells was assayed by incubating the AM-solubilized WLNCs (prepared as described in Example 2 at a polymer concentration of 15 μM and a WLNC concentration of 3.75 μM) diluted to 1 μM with respect to the polymer, with cells for 24 hours at 37° C. and 5% CO₂. The cells were then washed once with PBS and imaged for cell-associated fluorescence using a Nikon Eclipse TE2000-S.

Storage Stability of 1pM-solubilzed WLNCs. Four 15 mL samples of 1pM-solubilzed WLNCs as a polymer:WLNC ratio of 20 were prepared as described in Example 2. The four samples were distributed into three 5 mL aliquots then stored at four different storage conditions: 1) lyophilized on day 0 then stored at room temperature (˜25° C.), 2)-4° C. (freezer), 3) 2-8° C. (refrigeration), and 4) room temperature (˜25° C.). At day 0, 1, 2, 3, 7 and 14, the fluorescence of each of the samples was quantified as described in Example 2. For analysis, the three samples at each storage condition were averaged. For comparison, the fluorescence at 525 nm for each sample as a percentage of the original fluorescence (i.e., day 0) was graphed.

### Results

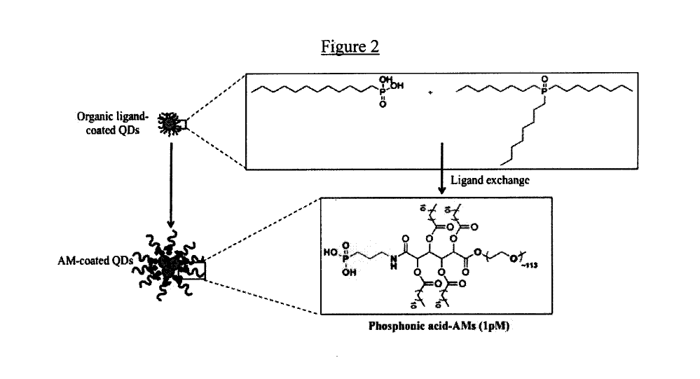

Water-solubilization of WLNCs with AMs. Two polymers were employed to water-solubilize WLNC: a carboxylic acid-terminated AM, 1cM, and a phosphonic acid-terminated AM, 1pM. The 1cM was utilized to encapsulate the WLNC while the 1pM was modified from 0cM to chemically incorporate a phosphonic acid moiety. Specifically, 1pM was synthesized from 0cM and 3-aminopropylphosphonic acid, as shown below. The 1pM was designed to ligand exchange with the organic ligands, TOPO and octadodecylphosphonic acid, on the WLNC surface, shown in FIG. 2 (A. F. E. Hezinger, J. Tebmar, A. Gopferich, European Journal of Pharmaceutics and Biopharmaceutics 2008, 68, 138; M. J. Bowers II, J. R. McBride, S. J. Rosenthal, Journal of the American Chemical Society 2005, 127, 15378).

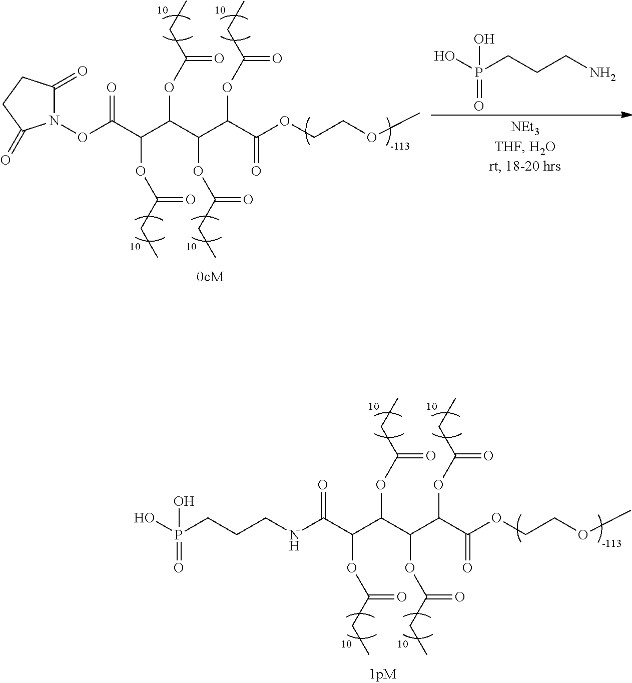

Synthesis of 1pM was verified by proton nuclear magnetic resonance (¹H NMR) spectroscopy and molecular weight determined by gel permeation chromatography (GPC) relative to PEG standards. Due to the abundance of PEG in the polymer (˜83% of protons), the presence of new protons in the ¹H NMR spectra were difficult to detect, particularly from 0.8 to 2.4 ppm where the methylene protons of the hydrophobic chains comprise the majority of that region. Thus, spectra were monitored for the disappearance of the protons of the activating group (N-hydroxysuccinimide) on 0cM, which resonate at 2.8 ppm.

Following the successful synthesis of 1pM, both 1cM and 1pM were employed to water-solubilize the WLNCs using a solvent evaporation method. Examples of variations on solvent evaporation methods used to water-solubilize fluorescent nanocrystals are described in the following documents: A. M. Smith, H. Duan, M. N. Rhyner, G. Ruan, S, Nie, Physical Chemistry Chemical Physics 2006, 8, 3895; D. L. Nida, N. Nitin, W. W. Yu, V. L. Colvin, R. Richards-Kortum, Nanotechnology 2008, 19, 1; W. W. Yu, E. Chang, J. C. Falkner, J. Zhang, A. M. Al-Somali, C. M. Sayes, J. Johns, R. Drezek, V. L. Colvin, Journal of the American Chemical Society 2007, 129, 2871; E. E. Lees, T.-L. Nguyen, A. H. A. Clayton, P. Mulvaney, ACS Nano 2009, 3, 1121. The method used in this Example was carried out by dissolving the polymers and the WLNCs in a volatile, organic cosolvent (i.e., chloroform) and the solutions agitated in foil-covered vials for three hours. The chloroform was then removed by rotary evaporation and the resulting yellow films dried overnight (can be left as films longer) before re-dissolving in water by sonication.

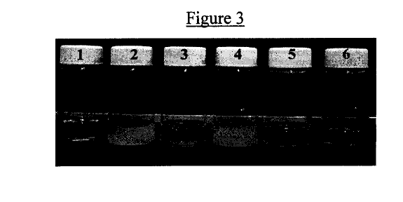

Characterization of Water-soluble WLNCs. Water-solubilization of the WLNCs was determined by visual inspection of the resulting solutions, as shown in FIG. 3. Solubilized nanocrystals were uniformly dispersed throughout the yellow solution, while unsolubilized nanocrystals deposited on the vial walls, as in the QD in water control. Both 1cM and 1pM were successful at solubilizing the WLNCs. The WLNCs in 1cM solutions appeared cloudy and turbid, while WLNCs in 1pM were more transparent.

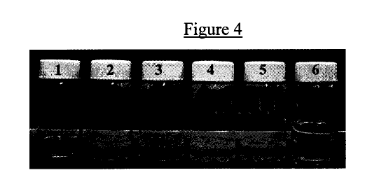

The solutions were then filtered with a 0.45 μm syringe filter to remove the larger particles. It was often necessary to use multiple filters when one became clogged, i.e. to filter 5 mL of sample 4-5 individual filters were necessary to filter the 1pM-solubilized WLNCs where as 15-17 individual filters were necessary to filter the 1cM-solubilized WLNCs. The use of more filters for the same sample volume indicates there are more assemblies larger than the filter size (i.e., 450 nm) in the 1cM-solubilized WLNC solutions. Qualitatively, as shown in FIG. 4, the filtered solutions of WLNC with 1cM in H₂O contained far less nanocrystals judging by the decrease in yellow color and solution opacity. In contrast, the WLNC solutions with 1pM in H₂O are visually similar to the unfiltered samples (FIGS. 3 and 4).

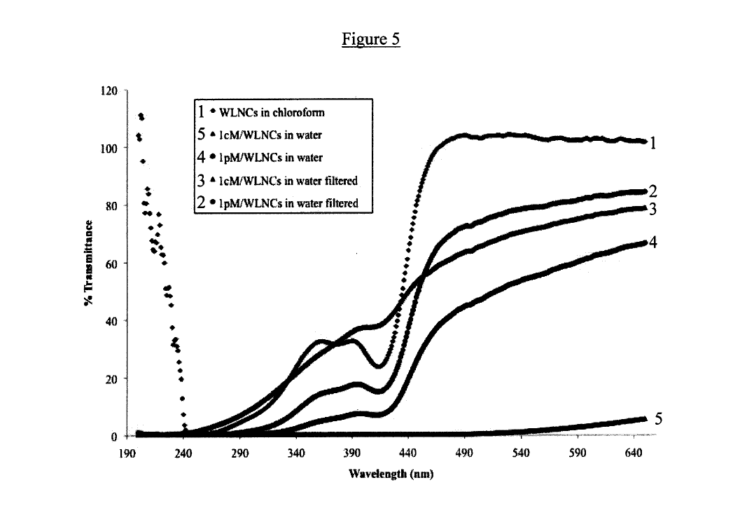

Turbidity. Turbidity was quantitatively determined using UV/Vis absorbance spectroscopy, shown in FIG. 5. WLNCs dispersed in chloroform were tested as a standard for the amount of light that could be transmitted in solutions without turbidity, as the light transmitted is only that light which was not absorbed by the nanocrystals. Confirming the qualitative visual inspection, the WLNC samples with 1cM dispersed in H₂O resulted in 0-6% transmittance from 190-600 nm, while the WLNC solutions with 1pM dispersed have a much higher % transmittance from 340-640 nm, with a curve similar to the WLNCs in chloroform. Following filtration, samples of WLNCs dispersed in water with both 1cM and 1pM have significantly higher light transmittance across the spectra compared to the samples without filtration. However, while both samples of 1pM/WLNCs, filtered and unfiltered, have transmittance curves with features similar to the WLNCs in chloroform (i.e. peaks ˜360, 390, and an approx. leveling off at ˜490 nm), only the filtered sample of 1cM/WLNCs has a curve that modestly resembles that of the WLNCs in chloroform.

Overall, the percent transmittance data correlates with the qualitative imaging results in that the samples of 1pM-solubilized WLNCs are less turbid than the 1cM-solubilized WLNCs. However, when the 1cM-solubilized WLNC solution is filtered, the turbidity decreases significantly. Additionally, this data shows that the 1pM-solubilized WLNCs maintain transmittance spectra more similar to the WLNCs in chloroform than do those nanocrystals water-solubilized using 1cM.

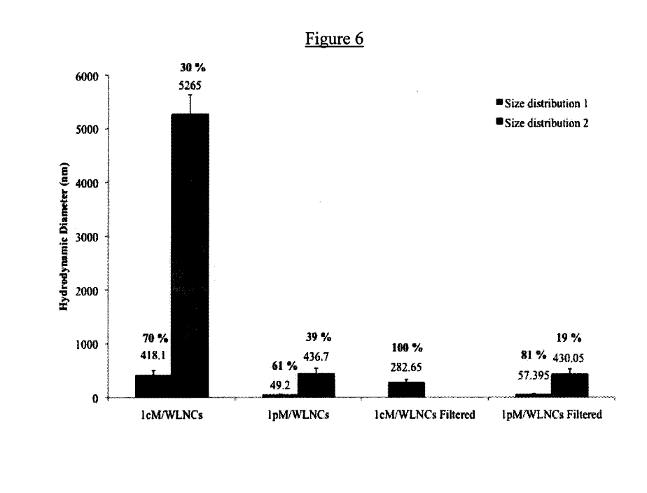

Assembly Sizes. Hydrodynamic diameters of the aqueous WLNC solutions were determined using dynamic light scattering, the results are shown in FIG. 6. For the unfiltered samples, the assemblies and/or aggregates of WLNC with each polymer existed as two distinct size distributions, with the smaller sized particles as the major solution component. In comparing the assemblies and/or aggregates of the 1cM-solubilized WLNC (1cM/WLNCs) to the 1pM-solubilized WLNC (1pM/WLNCs), ˜60% of the 1pM-solubilized WLNC assemblies are ˜49 nm while the other ˜40% are ˜435 nm in diameter. In contrast, the larger sized particles of the 1cM-solubilized WLNCs (˜420 nm) account for 70% of the volume, while the remaining 30% is attributed to 5 μm size assemblies/aggregates.

Upon filtering the samples with a 0.45 μm syringe filter, the larger aggregates are greatly reduced as observed by DLS in FIG. 6 and in accordance with the visual results, FIG. 4. For the 1cM-solubilized WLNCs, the 5 gm assemblies/aggregates are completely removed and the remaining assemblies form sizes of ˜280 nm, indicating that mechanical forces of the filter break up the larger aggregates. Conversely, the 1pM-solubilized WLNC still exist as two size distributions of 57 nm and 430 nm. However, the percentage of the smaller sized particles increased by about 20%. This result again suggests that the mechanical force of filtration reduces the aggregate size. As a whole, these results are consistent with the turbidity data; the assemblies from 1cM-solubilized WLNC are larger and transmit significantly less light than 1pM-solubilized WLNC. Further, the qualitative results visualized before and after sample filtering (FIG. 3 and FIG. 4) demonstrated that turbidity of the 1cM-solubilized WLNC samples significantly decreased following filtration (ie, larger aggregates and assemblies were removed and/or disassociated) while minor changes were observed with 1pM-solubilized WLNC.

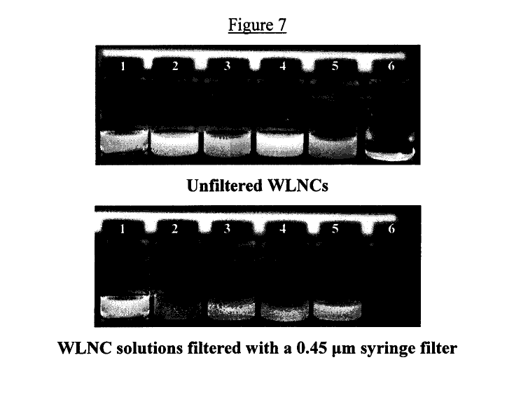

Qualitative Fluorescence Emission. Fluorescence emission was qualitatively determined by excitation with a long wavelength UV lamp at 365 nm. As shown in FIG. 7 (top), All solutions except the WLNC dispersed in water only emit bright white light that is evenly dispersed throughout the solution, indicating successful solubilization. For WLNC samples in water, the WLNC adheres to the vial walls, indicating a lack of aqueous solubilization of the hydrophobic nanocrystals.

When the solutions were filtered with a 0.45 μm syringe filter, the white light fluorescence intensity for the aqueous WLNC solution with 1cM significantly decreased compared with the unfiltered solutions, whereas the aqueous WLNC solution with 1pM fluoresces as intensely as the unfiltered samples (shown in FIG. 7 (bottom)). This observation indicates that the majority of the nanocrystals with 1cM exist as assemblies or aggregates larger than 450 nm. However, the fluorescence intensity of the nanocrystals with 1pM have a qualitatively similar fluorescence intensity, suggesting the majority of the assemblies and/or aggregates are smaller than 450 nm.

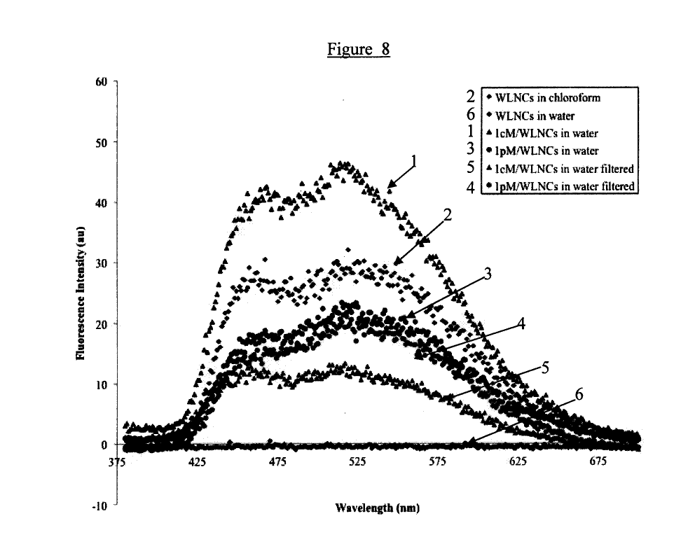

Quantitative Fluorescence Emission. Quantitative fluorescence emission data for all samples was collected using fluorescence spectroscopy from 380-800 nm following excitation at 365 nm (shown in FIG. 8).

All solutions of WLNC, with the exception of the nanocrystals in water only, shared a similar fluorescence profile (i.e., broadband white light-emission and similar λₘₐₓ peaks) as the nanocrystals dissolved in chloroform at the same concentration. The 1cM-solubilized WLNCs (1cM/WLNCs in water) had fluorescence intensity significantly greater than the nanocrystals in chloroform (approximately 1.5 times greater) while the 1pM-solubilized WLNCs (1pM/WLNCs in water) had a fluorescence emission approximately 70% that of the nanocrystals in chloroform. When filtered, the 1pM-solubilized WLNC (1pM/WLNC in water filtered) had approximately the same fluorescence intensity as the unfiltered sample across the spectrum. In contrast, the filtered sample of 1cM-solubilized WLNCs (1cM/WLNC in water filtered) lost approximately 75% of the fluorescence of the unfiltered sample, dropping to ˜30% of the nanocrystal fluorescence in chloroform at the original concentration. This quantitative fluorescence data is consistent with visual inspection (FIG. 7).

Taken together with the sizing data, the 1cM-solubilized WLNC data suggests that the majority of the solution fluorescence results from the assemblies/aggregates of 5 μm in size. This phenomenon is interesting, as these large particles only account for ˜30% of the overall size distribution for the sample. As the fluorescence intensity of this aqueous solution is greater than nanocrystals dissolved in chloroform at the same concentration, the data suggests that nanocrystal aggregations improve their fluorescence intensity compared to smaller sized assemblies. As the goal was to create systems that are small assemblies with fluorescence unaffected by water-solubilization, the 1pM-solubilized WLNCs have the optimal balance of small size distributions and largely unaffected fluorescence emission profiles.

It should also be noted that a large emission peak was also observed for all AM-solubilized WLNC solutions at ˜728 nm. This NIR emission peak can be attributed to the second diffraction peak from the excitation source passing through slits and a grating, which occurs when the sample is slightly scattering the excitation light.

Preliminary Cellular Uptake. The ability of water-soluble WLNCs to be internalized was evaluated in THP-1 macrophage cells after 24 hour incubation. The cells were imaged at 20× magnification using multiple microscope filters to show retention of white-light emission. Both 1cM-solubilized WLNCs and 1pM-solubilized WLNCs are internalized by cells and continue to emit white light. In comparing the two polymers for solubilizing the WLNCs, it is clear that 1pM-solubilized WLNCs are more effectively internalized due to the larger number of fluorescent cells and their higher intensity. In addition, cells incubated with 1pM-solubilized WLNCs under the UV filter with increased magnification (40×) show the 1pM-solubilized WLNCs appear to be localized within the nuclei of the cells (excitation: 340-380 nm, emission: 435-485 nm). The cells appeared healthy, indicating the assemblies exhibited low cytotoxicity; biocompatibility is often a barrier to using fluorescent QDs for biological applications (R. J. Martin-Palma, M. Manso, V. Tones-Costa, Sensors 2009, 9, 5149). This data shows that for cellular applications, 1pM-solubilized WLNCs are more efficient, which is likely due to the smaller assembly size of these systems as compared to the 1cM-solubilized WLNCs.

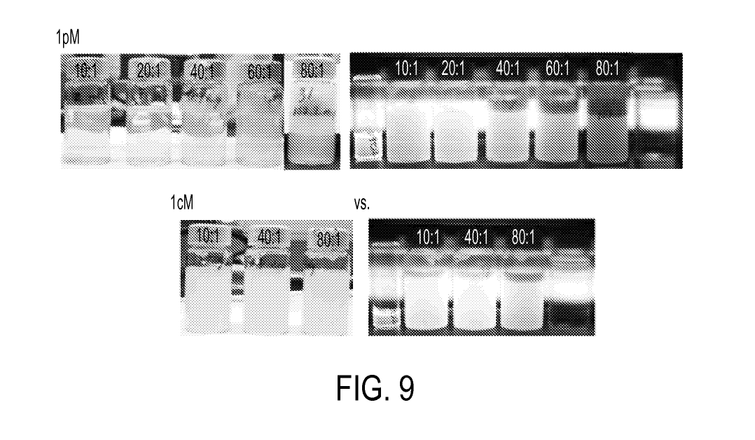

Evidence for Ligand Exchange Using 1pM. Hitherto, the 1pM is referenced as a polymer capable of coordinating to the nanocrystal surface. To investigate ligand exchange mechanisms, increasing polymer concentrations with respect to the nanocrystals was investigated; as more polymer becomes available to exchange the surface organic ligands, the WLNC fluorescence properties would be modified. As qualitatively shown in FIG. 9, increasing the polymer:WLNC ratio from 10 to 80 gradually shifted the fluorescence emission from white to blue light. This data indicates increased interactions of the ligands with the QD surface resulting in altered fluorescence. Some potential causes are: 1) the ligands create additional surface defects causing excited electrons to be “captured” before emitting their energy radiatively, or 2) ligand interaction with the uncoordinated surface selenium atoms decreasing the ability of the selenium to participate in deep-trap emission. Thus, in the case of the 1pM-solubilized WLNCs, the shift from white-light emission to the emission of blue light suggests that those electrons relaxing through higher energy pathways continue to emit light while low energy electrons become trapped by ligands and/or relax through different, non-radiative pathways.

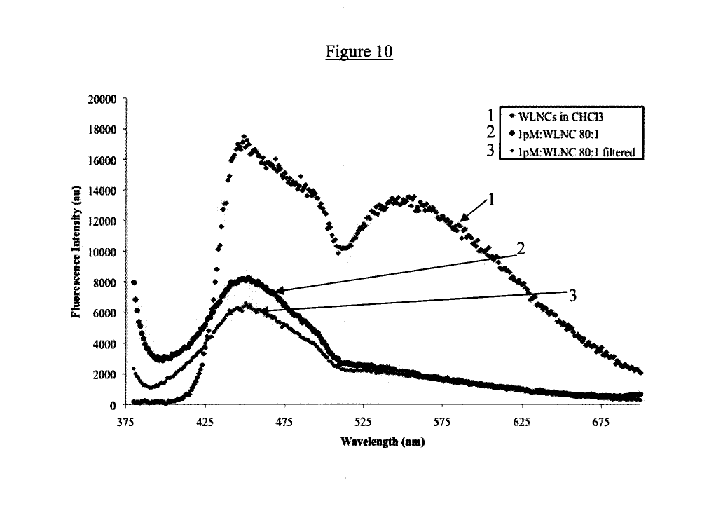

This change is shown quantitatively in FIG. 10 for the 1pM:WLNC ratio of 80:1 in which the peak at 524 nm substantially decreases relative to the unmodified nanocrystals in chloroform. When the ratio of 1cM:WLNC is increased from 10 to 80, this shift from white to blue light emission by visual inspection in FIG. 9 is not observed. This result supports the hypothesis that increasing the concentration of 1pM results in increased interactions between the polymer and nanocrystal surface. The increased polymer-WLNC interactions result in significant changes in fluorescence emission, from white-light to blue light.

Storage Stability of 1pM-solubilized WLNC Assemblies. As discussed earlier, an optimal polymer to water solubilize the WLNCs would have the capability to water-solubilize the WLNCs into assemblies that maintain the nanocrystal size and fluorescence properties. Based upon the previously discussed results, the 1pM-solubilized WLNCs were determined to be the better polymer over 1cM—with small size distributions and largely unaffected fluorescence emission profiles. Therefore, only the 1pM-solubilized WLNCs were evaluated for storage stability.

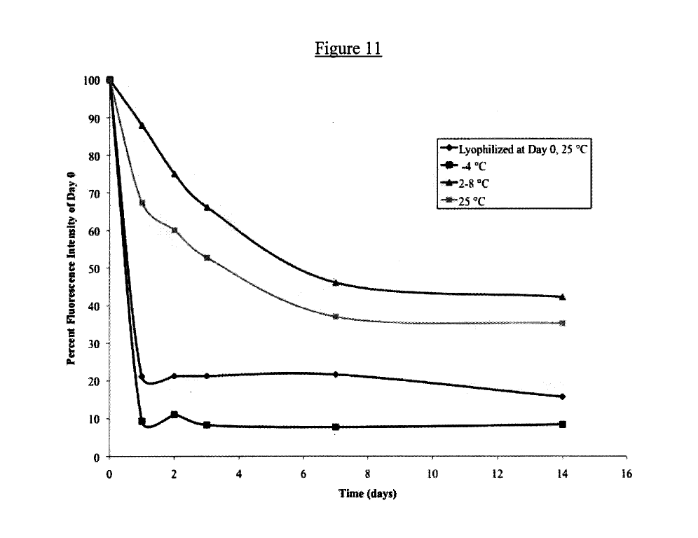

To assess the appropriate storage conditions and stability of the 1pM-solubilized WLNCs, samples at a polymer:nanocrystal ratio of 20:1 were tested for fluorescence emission over two weeks. The storage conditions included: a lyophilized condition (samples frozen and lyophilized on Day 0, then resolubilized and stored at −4° C. for the remainder of the two weeks); and solutions stored at −4° C. (freezer), 2-8° C. (refrigerator), and 25° C. (room temperature). Fluorescence emission data was collected from 380-800 nm following excitation at 365 nm. The data was then analyzed and the sample fluorescence intensity at 525 nm with respect to the original fluorescence (i.e., day 0) graphed in FIG. 11.

After 24 hours, the fluorescence intensity for all samples significantly decreased, but less so for solutions stored at 2-8° C. and 25° C. After the first freeze-thaw cycle, samples stored at −4° C. retained 10% of their original intensity, while solutions stored at 2-8° C. or 25° C. continually decreased over fourteen days to ˜40% of their original fluorescence intensity. While the data shown is only for the emission peak at 525 nm, the fluorescence intensity for all samples at the emission peak at ˜450 nm similarly decreased, indicating an overall quenching of fluorescence properties. This data suggest that the first freeze-thaw cycle for nanocrystals water-solubilized using 1pM has detrimental effects on the nanocrystal fluorescence, since after the first freeze-thaw cycle the fluorescence intensity remains constant regardless of subsequent freezer storage. Thus, solutions are best stored at refrigeration or room temperatures; however, intensity does significantly decrease with storage time.

In summary, water-solubilization of WLNCs was achieved. Encapsulation using the carboxy-terminated AM (1cM) resulted in WLNCs with the most intense fluorescence, even greater than the original WLNCs dispersed in chloroform at the same concentration. However, smaller sizes were achieved by solubilizing the WLNCs with the functionalized AMs (1pM). Additionally, the sizes and fluorescence intensities were maintained for 1pM-solubilized WLNCs following filtration through a 0.45 μm syringe filter, while the intensity of fluorescence emission for the 1cM-solubilized WLNCs decreased to 75% of its original intensity. In addition, evidence of ligand exchange was determined qualitatively; increased polymer concentrations resulted in a shift from white- to blue-light emission, indicating the surface of the WLNCs had been altered. Based on the disappearance of emission above 500 nm, the surface modification caused excited electrons with low energy-emission to relax non-radiatively. Finally, the storage stability of the ligand exchange-solubilized systems was evaluated over fourteen days at a range of temperatures and it was determined that freshly solubilized samples displayed optimal fluorescence properties. However, if sample storage was necessary, refrigeration is best.

### Example 2

In this Example, a series of polymers with varying functional groups were evaluated to biostabilize WLNCs with the same goals as Example 1: maintenance of WLNC size and white-light fluorescence emission.

To optimize the AM-solubilized WLNCs, a variety of functional groups were chemically incorporated within the hydrophobic portion of the polymer backbone. Functional groups included acidic groups, such as sulphate and phosphate, basic moieties, such as amines and hydroxyls, and soft ligands, such as organophosphine and thiol. The soft ligands were chosen as they are known to coordinate well to soft metals (i.e., cadmium) based on hard-soft acid base chemistry. The AMs with varying functional groups are shown below (p is ˜113), and for clarity will be described as the hydrophobic functionality that is intended to interact with the nanocrystal surface. For two functionalities, phosphonic acid and hydroxyl, multiple lengths of the alkane spacer were utilized to elucidate steric effects imparted by the AM's alkylated arms which may limit the ability of the functional group to coordinate to the nanocrystal surface. For the four polymers, the number of carbons in the alkane spacer is indicated in parenthesis following the functionality to differentiate the two. As an example, phosphonic acid (propyl) indicates a three-carbon spacer between the phosphonic acid and the amide bond to the polymer where as phosphonic acid (butyl) indicates a four-carbon space between the phosphonic acid and the amide bond to the polymer. The N-hydroxysuccinimide (NHS)-terminal AM, the carboxylic acid-terminal AM, the phosphonic acid (propyl)-terminal AM, and the sulfate-terminal AM are referenced as 0cM, 1cM, 1pM, and 1sM, respectively. The 0cM and 1cM AMs were used as controls for polymers not anticipated to coordinate to the nanocrystals, while 1pM was the standard, as it was shown in Example 1 to be more efficient at solubilizing the WLNCs based upon polymer interaction with the nanocrystal surface.

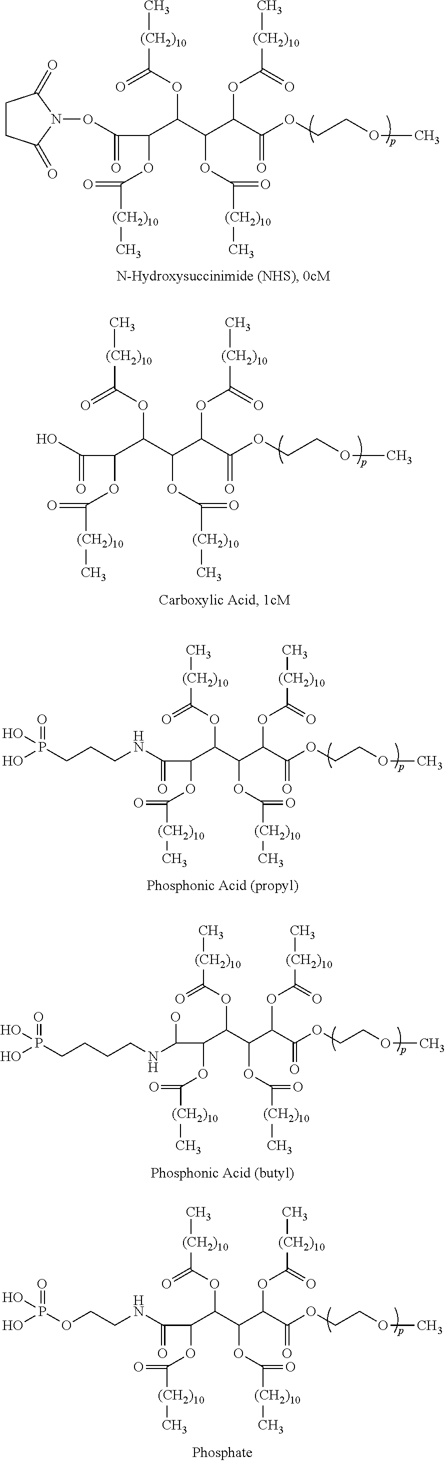

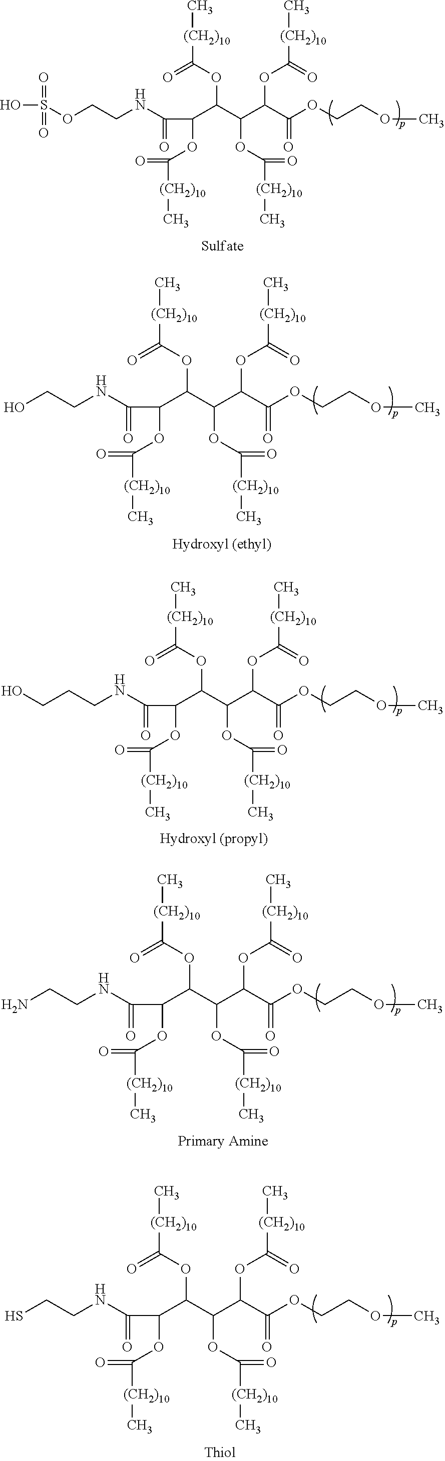

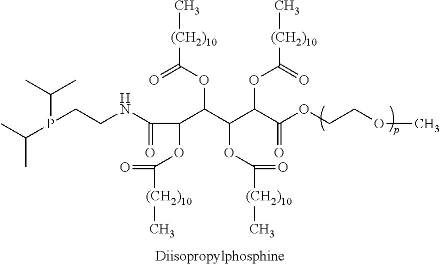

Similar to Example 1, the goal of this Example is to efficiently solubilize the WLNCs into small, nanoscale assemblies that still emit white-light. The phosphonic acid moieties were anticipated to be effective, as shown in Example 1. Other moieties that were expected to be efficient towards coordination, and therefore, water-solubilization were the soft ligands (i.e., thiol and diisopropylphosphine) due to the nature of their interaction with cadmium, a soft metal.

### Materials and Methods

Synthetic Materials. Unless otherwise stated, solvents and reagents were purchased from Fisher Scientific (Pittsburgh, Pa.) and Sigma-Aldrich (St. Louis, Mo.) and used as received. PEG 5 kDa was purchased from Polysciences, Inc. (Warrington, Pa.) and dried by azeotropic distillation from toluene before use. The carboxylic acid-terminal AM (1cM) and 0cM (starting material for all functionalized AMs) were synthesized as previously described (J. Djordjevic, L. Del Rosario, J. Wang, K. Uhrich, Journal of Bioactive and Compatible Polymers 2008, 23, 532; L. Tian, L. Yam, N. Zhou, H. Tat, K. Uhrich, Macromolecules 2004, 37, 538). The synthesis of the phosphoric acid (propyl)-terminal AM was previously described in Example 1. The CdSe WLNCs were synthesized and provided as solutions in chloroform by Professor Sandra Rosenthal's group at Vanderbilt University (M. J. Bowers II, J. R. McBride, S. J. Rosenthal, Journal of the American Chemical Society 2005, 127, 15378).

Proton Nuclear Magnetic Resonance (¹H NMR) Spectroscopy. Proton nuclear magnetic resonance (¹H-NMR) spectra of the products were obtained using a Varian 400 MHz or 500 MHz spectrophotometer. Samples were dissolved in chloroform-d, with a few drops of dimethyl sulfoxide-d₆ if necessary, with tetramethylsilane as an internal reference.

Gel Permeation Chromatography (GPC). Molecular weights (M_{w}) and polydispersity indices (PDI) were determined using gel permeation chromatography (GPC) with respect to PEG standards (Sigma-Aldrich) on a Waters Stryagel® HR 3 THF column (7.8×300 mm). The Waters LC system (Milford, Mass.) was equipped with a 2414 refractive index detector, a 1515 isocratic HPLC pump, and 717plus autosampler. An IBM ThinkCentre computer with Waters Breeze Version 3.30 software installed was used for collection and processing of data. Samples were prepared at a concentration of 10 mg/mL in tetrahydrofuran, filtered using 0.45 μm pore size nylon or poly(tetrafluoroethylene) syringe filters (Fisher Scientific) and placed in sample vials to be injected into the system.

Polymer Synthesis: Sulfate-terminal AM. 2-Aminoethyl hydrogen sulfate (7.0 mg, 0.050 mmol) was dissolved in DMSO (2 mL) by warming over medium heat on a stir plate for 15-30 min. After cooling to room temperature, 0.5 M NaOH (101 μL) was added and the solution stirred for 30 min. In a separate flask, 0cM (0.20 g, 0.033 mmol) was dissolved in CH₂Cl₂ (6.0 mL) and subsequently added to the solution of 2-aminoethyl hydrogen sulfate dropwise and the reaction stirred overnight (12 hrs). The CH₂Cl₂ was then removed via rotary evaporation then the DMSO removed via lyophilization. The resulting solid was dissolved in CH₂Cl₂ and the solution filtered to remove excess 2-aminoethyl hydrogen sulfate and the N-hydroxysuccinimide by-product. The desired product was precipitated from CH₂Cl₂ by addition of 10-fold diethyl ether and the solid collected by centrifugation. Solvent was removed by decanting and the resulting yellow solid was dried under ambient atmosphere (12 hrs) and under high vacuum (12 hrs). Yield: 0.16 g, 78%. ¹H-NMR (CDCl₃): δ 5.83 (m, 2H, CH), 5.48 (m, 2H, CH), 3.67 (m, ˜0.45 kH, CH₂O), 3.38 (s, 3H, CH₃), 2.32 (m, 8H, CH₂), 1.60 (m, 8H, CH₂), 1.22 (m, 64H, CH₂), 0.88 (t, 12H, CH₃). Tₘ=55° C. GPC: M_{w}: 6.4 kDa; PDI: 1.08. A schematic is shown below.

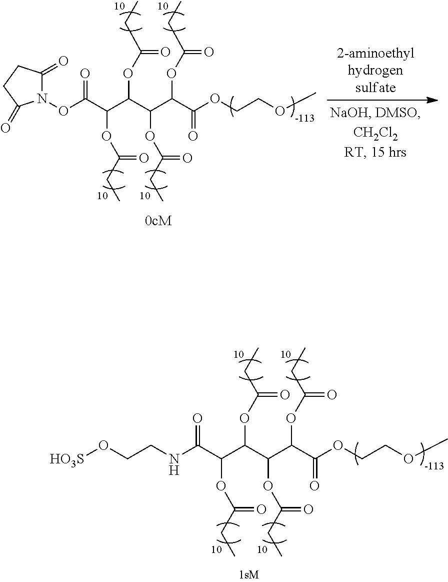

Polymer Synthesis: Primary amine-terminal AM. Ethylenediamine (50 μL, 0.75 mmol) was dissolved in HPLC-grade CH₂Cl₂ (3 mL) and triethylamine (0.15 mL, 1.1 mmol). In a separate vessel, 0cM (0.51 g, 0.085 mmol) was dissolved in HPLC-grade CH₂Cl₂ (9 mL) and subsequently added to the solution of ethylenediamine dropwise via syringe pump at a rate of 1.0 mL/hr. The reaction was stirred overnight (˜18 hrs). The reaction solution was then diluted with CH₂Cl₂ and subsequently washed with 0.1 N HCl/brine (1×) and brine (2×). The combined aqueous portions were extracted with CH₂Cl₂ and the combined organics dried over MgSO₄, and concentrated to a yellow oil. The desired product was precipitated from the oil dissolved in CH₂Cl₂ (5 mL) by addition of 10-fold diethyl ether and cooling over dry ice for 1 hr. The solid was then collected by centrifugation at 3000 rpm for 5 min and the supernatant removed by decanting. The resulting white solid was dried under ambient atmosphere (12 hrs) and under high vacuum (12 hrs). Yield: 0.41 g, 80%. ¹H-NMR (CDCl₃): δ 5.67 (m, 2H, CH), 5.14 (m, 2H, CH), 4.24 (m, 3H, CH₂), 3.60 (m, ˜0.45 kH, CH₂O), 3.37 (s, 3H, OCH₃), 2.37 (m, 8H, CH₂), 2.29 (m, 4H, CH₂), 1.81 (b, 4H, CH₂), 1.60 (m, 8H, CH₂), 1.26 (m, 64H, CH₂), 0.87 (t, 12H, CH₃). Tₘ=58° C. GPC: M_{w}: 6.3 kDa; PDI: 1.1.

Polymer Synthesis Phosphonic Acid (Butyl)-terminal AM. In a 50 mL round bottom flask, 4-aminobutyl phosphonic acid (58.7 mg, 0.383 mmol) was dissolved in HPLC-grade H₂O (4 mL), HPLC-grade THF (6.5 mL), and triethylamine (0.20 mL, 1.4 mmol) and the solution stirred. In a separate flask, 0cM (531 mg, 0.0885 mmol) was dissolved in HPLC-grade THF (6 mL) and the solution added to the reaction flask. The yellow solution was stirred for 18-20 hrs at room temperature before the THF was removed by rotary evaporation. The resulting yellow oil was dissolved in CH₂Cl₂ and 0.1 N HCl and stirred for 20-30 min. The mixture was then transferred to a separatory funnel containing addition 0.1 N HCl and the organic layer separated and washed with brine (2×). The combined aqueous portions were extracted with CH₂Cl₂ and the combined organics dried over MgSO₄ and concentrated to a yellow oil. White product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with ether (1×) and cold hexanes (1×). The white solid was dried under ambient conditions (24 hrs) and under high vacuum (24 hrs). Yield: 0.38 mg, 71%. ¹H-NMR (CDCl₃): δ 5.67 (m, 2H, CH), 5.20 (m, 2H, CH), 4.20 (m, 2H, CH₂), 3.66 (m, ˜0.45 kH, CH₂O), 3.39 (s, 3H, CH₃), 2.39 (m, 8H, CH₂), 1.58 (m, 10H, CH₂), 1.27 (m, 64H, CH₂), 0.89 (t, 12H, CH₃). GPC: Mw: 7.2 kDa; PDI: 1.1.

Polymer Synthesis: Phosphate-terminal AM. In a 50 mL round bottom flask, 4-aminopropyl dihydrogen phosphate (47 mg, 0.33 mmol) was dissolved in HPLC-grade H₂O (2.5 mL), HPLC-grade THF (5 mL), and triethylamine (0.16 mL, 1.2 mmol) and the solution stirred. In a separate flask, 0cM (451 mg, 0.0752 mmol) was dissolved in HPLC-grade THF (6.2 mL) and the solution added to the reaction flask. The yellow solution was stirred for 18-20 hrs at room temperature before the THF was removed by rotary evaporation. The resulting yellow oil was dissolved in CH₂Cl₂ and 0.1 N HCl and stirred for 20-30 min. The mixture was then transferred to a separatory funnel containing addition 0.1 N HCl and the organic layer separated and washed with brine (2×). The combined aqueous portions were extracted with CH₂Cl₂ and the combined organic portions dried over MgSO₄ and concentrated to a yellow oil. White product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with ether (1×) and cold hexanes (1×). The white solid was dried under ambient conditions (24 hrs) and under high vacuum (24 hrs). Yield: 0.30 g, 66%. ¹H-NMR (CDCl₃): δ 5.70 (m, 2H, CH), 5.19 (m, 2H, CH), 4.23 (m, 3H, CH₂), 3.67 (m, ˜0.45 kH, CH₂O), 3.39 (s, 3H, CH₃), 2.37 (m, 8H, CH₂), 1.65 (m, 13H, CH₂), 1.27 (m, 68H, CH₂), 0.89 (t, 12H, CH₃). GPC: Mw: 7.3 kDa; PDI: 1.1.

Polymer Synthesis: Hydroxyl(Ethyl)-terminal AM. In a 50 mL round bottom flask, ethanolamine (36.8 μL, 0.612 mmol) was dissolved in HPLC-grade CH₂Cl₂ (2.5 mL) and triethylamine (0.17 mL, 1.2 mmol) and the solution stirred. In a separate flask, 0cM (459 mg, 0.0765 mmol) was dissolved in HPLC-grade CH₂Cl₂ (7.7 mL) and the solution added to the reaction flask dropwise via syringe pump at a rate of 1.0 mL/hr. The reaction was stirred for 18-20 hrs at room temperature before the bright yellow solution was filtered to remove the white solid (NHS by-product) and the filtrate washed with 0.1 N HCl/brine (1×) and brine (2×). The combined aqueous portions were extracted with CH₂Cl₂ and the combined organic portions dried over MgSO₄ and concentrated to a yellow oil. White product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with ether (1×) and cold hexanes (1×). The white solid was dried under ambient conditions (24 hrs) and under high vacuum (24 hrs). Yield: 0.31 g, 68%. ¹H-NMR (CDCl₃): δ 5.68 (m, 2H, CH), 5.15 (m, 2H, CH), 4.19 (m, 4H, CH₂), 3.60 (m, ˜0.45 kH, CH₂O), 3.39 (s, 3H, CH₃), 2.38 (m, 8H, CH₂), 1.62 (m, 14H, CH₂), 1.27 (m, 64H, CH₂), 0.89 (t, 12H, CH₃). GPC: Mw: 7.4 kDa; PDI: 1.1.

Polymer Synthesis: Hydroxyl(Propyl)-terminal AM. In a 50 mL round bottom flask, 3-amino-1-propanol (46.8 μL, 0.612 mmol) was dissolved in HPLC-grade CH₂Cl₂ (2.5 mL) and triethylamine (0.17 mL, 1.2 mmol) and the solution stirred. In a separate flask, 0cM (0.46 g, 0.077 mmol) was dissolved in HPLC-grade CH₂Cl₂ (7.8 mL) and the solution added to the reaction flask dropwise via syringe pump at a rate of 1.0 mL/hr. The reaction was stirred for 18-20 hrs at room temperature before the bright yellow solution was filtered to remove the white solid (NHS by-product) and the filtrate washed with 0.1 N HCl (1×) and brine (2×). The combined aqueous portions were extracted with CH₂Cl₂ and the combined organic portions dried over MgSO₄ and concentrated to a yellow oil. White product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with diethyl ether (1×) and cold hexanes (1×). The white solid was dried under ambient conditions (24 hrs) and under high vacuum (24 hrs). Yield: 0.34 g, 74%. ¹H-NMR (CDCl₃): δ 5.68 (m, 2H, CH), 5.21 (m, 2H, CH), 4.26 (m, 4H, CH₂), 3.67 (m, ˜0.45 kH, CH₂O), 3.39 (s, 3H, CH₃), 2.35 (m, 8H, CH₂), 1.65 (m, 25H, CH₂), 1.27 (m, 57H, CH₂), 0.89 (t, 12H, CH₃). GPC: Mw: 7.4 kDa; PDI: 1.1.

Polymer Synthesis: Thiol-terminal AM. In a 50 mL round bottom flask, cysteamine (30.5 mg, 0.395 mmol) was dissolved in HPLC-grade H₂O (1 mL), HPLC-grade THF (6.9 mL), and triethylamine (0.17 mL, 1.2 mmol) and the solution stirred. In a separate flask, 0cM (0.475 g, 0.0791 mmol) was dissolved in HPLC-grade THF (7.5 mL) by warming to 37° C. The clear, yellow solution was then added to the reaction flask. The clear, yellow solution was stirred for 19 hrs before the THF was removed by rotary evaporation. The resulting yellow solid was dissolved in CH₂Cl₂ and 0.1 N HCl and stirred for 20-30 min. The mixture was then transferred to a separatory funnel containing addition 0.1 N HCl and the organic layer separated and washed with brine (2×). The combined aqueous portions were extracted with CH₂Cl₂ and the combined organic portions dried over MgSO₄ and concentrated to a yellow oil. White product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with ether (1×) and cold hexanes (1×). The white solid was dried under ambient conditions (24 hrs) and under high vacuum (48 hrs). Yield: 0.39 g, 82%. ¹H-NMR (CDCl₃): δ 5.63 (m, 2H, CH), 5.15 (m, 2H, CH), 4.27 (m, 2H, CH₂), 3.65 (m, ˜0.45 kH, CH₂O), 3.39 (s, 3H, CH₃), 2.38 (m, 8H, CH₂), 1.75 (m, 4H, CH₂), 1.58 (m, 8H, CH₂), 1.27 (m, 64H, CH₂), 0.89 (t, 12H, CH₃). GPC: Mw: 7.2 kDa; PDI: 1.1.

Polymer Synthesis: Diisopropylphosphine-terminal AM. In a 50 mL round bottom flask, 2-(diisopropylphosphino)ethyl amine (0.114 g, 0.709 mmol) was dissolved in anhydrous CH₂Cl₂ (2.8 mL) and triethylamine (0.19 mL, 1.4 mmol) and the solution stirred. In a separate flask, 0cM (0.431 g, 0.0861 mmol) was dissolved in anhydrous CH₂Cl₂ (8.7 mL) and the solution added to the reaction flask dropwise via syringe pump at a rate of 1.0 mL/hr. The reaction was stirred for 24 hrs at room temperature before the yellow solution was washed with 0.1 N HCl (1×) and brine (2×). The combined aqueous portions were extracted with CH₂Cl₂ and the combined organic portions dried over MgSO₄ and concentrated to a yellow oil. A beige product was precipitated from the yellow oil in CH₂Cl₂ by addition of 10-fold diethyl ether in a 50 mL centrifuge tube. The suspension was place on a shaker for 10-15 min before the solid was collected by centrifugation and the supernatant removed by decanting. The solid was dissolved in CH₂Cl₂ (<5 mL) and reprecipitated by adding 10-fold diethyl ether. The solid was collected by centrifugation, the supernatant removed, and the solid washed with ether (1×) and cold hexanes (1×). The white solid was dried under ambient conditions (24 hrs) and under high vacuum (24 hrs). Yield: 0.36 g, 69%. ¹H-NMR (CDCl₃): δ 5.66 (m, 2H, CH), 5.15 (m, 2H, CH), 4.22 (m, 2H, CH₂), 3.60 (m, ˜0.45 kH, CH₂O), 3.38 (s, 3H, CH₃), 2.38 (m, 8H, CH₂), 1.99 (m, 3H, CH₃), 1.80 (m, 3H, CH₃), 1.50 (m, 8H, CH₂), 1.23 (m, 64H, CH₂), 0.89 (t, 12H, CH₃). GPC: Mw: 7.4 kDa; PDI: 1.1.

Water-solubilization of WLNCs by Solvent Evaporation. Water-solubilization was performed using the solvent evaporation method detailed in Example 1, using a WLNC stock solution in chloroform with a concentration of 0.660 μM (determined as previously described in W. W. Yu, L. Qu, W. Guo, X. Peng, Chemistry of Materials 2003, 15, 2854), a final volume during stirring of 3.2 mL, and the specific amounts described in Table 3 below.

TABLE 3
Specific amounts and concentrations of functionalized AMs and
WLNCs used to formulate the water-solubilized nanocrystals.

| | | | | Stirring | | Water-Soluble WLNCs | | |
|---|---|---|---|---|---|---|---|---|
| Functionalized | AM | AM | WLNCs | [AM] | [WLNC] | [AM] | [WLNC] | |
| AM | (mg) | (μmol) | (μmol) | (μM) | (μM) | (μM) | (μM) | AM:WLNC |
| NHS | 19.9 | 3.32 | 0.165 | 1040 | 51.6 | 663 | 33.0 | 20.1 |
| Carboxylic Acid | 19.9 | 3.32 | 0.165 | 1040 | 51.6 | 663 | 33.0 | 20.1 |
| Phosphonic Acid | 20.4 | 3.34 | 0.165 | 1050 | 51.6 | 669 | 33.0 | 20.3 |
| (Propyl) | | | | | | | | |
| Phosphonic Acid | 19.9 | 3.26 | 0.165 | 1020 | 51.6 | 653 | 33.0 | 19.8 |
| (Butyl) | | | | | | | | |
| Phosphate | 19.9 | 3.26 | 0.165 | 1020 | 51.6 | 653 | 33.0 | 19.8 |
| Sulfate | 20.8 | 3.41 | 0.165 | 1070 | 51.6 | 682 | 33.0 | 20.7 |
| Primary Amine | 20.8 | 3.47 | 0.165 | 1080 | 51.6 | 693 | 33.0 | 21.0 |
| Hydroxyl (Ethyl) | 20.5 | 3.42 | 0.165 | 1070 | 51.6 | 683 | 33.0 | 20.7 |
| Hydroxyl (Propyl) | 20.2 | 3.37 | 0.165 | 1050 | 51.6 | 673 | 33.0 | 20.4 |
| Thiol | 19.6 | 3.27 | 0.165 | 1020 | 51.6 | 653 | 33.0 | 19.8 |
| Diisopropyl- | 19.9 | 3.32 | 0.165 | 1040 | 51.6 | 663 | 33.0 | 20.1 |
| phosphine | | | | | | | | |

Qualitative Evaluation of Turbidity and Fluorescence. Turbidity of water-solubilized WLNCs as compared with WLNCs in chloroform at the same concentration was qualitatively evaluated by visual inspection and images captured using a Canon PowerShot SD400 digital camera. Fluorescence of the solutions was qualitatively evaluated following excitation with a Spectroline® Longlife™ Filter long wavelength UV lamp (365 nm) and images captured using a Canon PowerShot SD400 digital camera.

Quantitative Turbidity: Percent Transmittance. Turbidity of water-solubilized WLNCs as compared with WLNCs in chloroform at the same concentration was evaluated quantitatively by UV/Visible absorbance on a Lambda Bio XLS instrument (Perkin Elmer, Waltham, Mass.) scanning from 190-650 nm.

Quantitative Fluorescence Emission. Fluorescence of water-solubilized WLNCs and WLNCs in chloroform at the same concentration from 380-800 nm was quantified using a Shimadzu RF-5301 PC spectrofluorophotometer, with an excitation wavelength of 365 nm.

Hydrodynamic Diameter. Hydrodynamic diameters were evaluated by dynamic light scattering (DLS) using a Malvern Instruments Zetasizer Nano ZS-90 instrument (Southboro, Mass.). DLS measurements were performed at a 90° scattering angle at 25° C. Size distributions by volume of measurements were collected in triplicate, averaged and reported.

### Results

Polymer Synthesis & Water-solubilizaton. Synthesis of AMs with the varying hydrophobic functionalities, shown above, were carried out using one of the two methods shown below, dependent upon the physical state of the amino starting material. For liquid staring materials (i.e., primary amine, diisopropylphosphine, and both hydroxyl-terminal AMs), the compounds were added to the NHS-terminal AM (0cM) using Scheme B. For solid starting materials (i.e., phosphate, sulfate, thiol, and both phosphonic acid-terminal AMs), the compounds were added to the NHS-terminal AM (0cM) using Scheme A, as they were insoluble in methylene chloride and all polar organic solvents that are miscible with methylene chloride but were water-soluble. Tetrahydrofuran (86% v/v) was added to the solutions to ensure no significant micelle formation resulting from the water-solubilization; it has previously been shown that addition of 80% v/v organic solvent to the polymers in water was sufficient to disrupt micelle formation (data not shown).

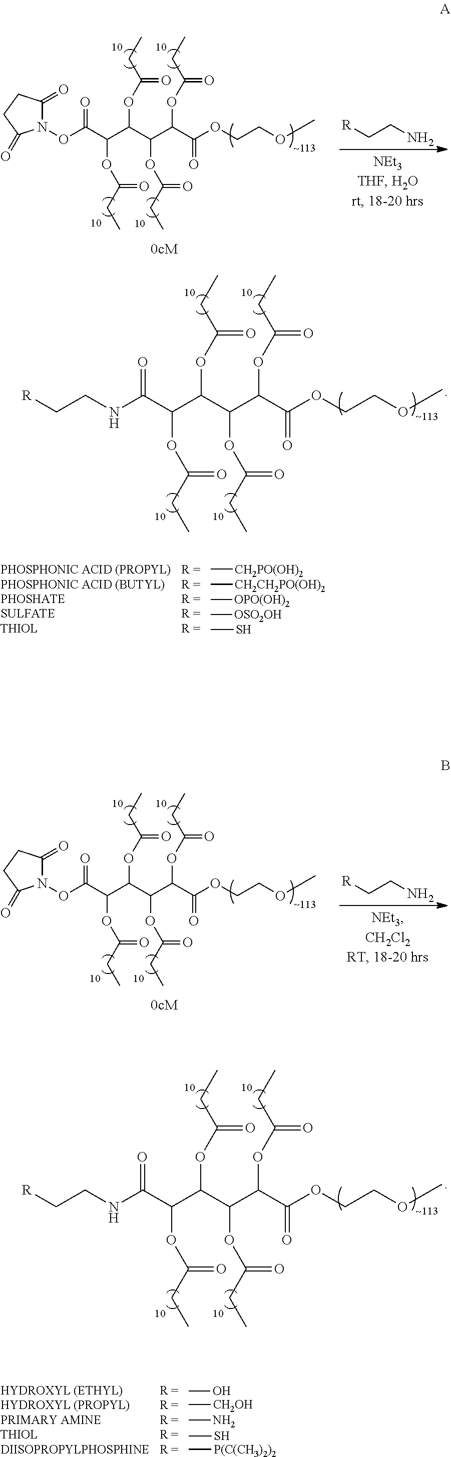

Successful synthesis of the polymers was verified by proton nuclear magnetic resonance (¹H NMR) spectroscopy and molecular weight determined by gel permeation chromatography (GPC) relative to PEG standards. Due to the abundance of PEG in the polymer (˜83% of protons), the presence of new protons in the ¹H NMR spectra were difficult to detect, particularly from 0.8 to 2.4 ppm where the methylene protons of the hydrophobic chains comprise the majority of that region. Thus, spectra were monitored for the disappearance of the protons of the activating group (N-hydroxysuccinimide) on 0cM, which resonate at 2.8 ppm.

Following synthesis, the polymers were utilized to water-solubilize the WLNCs, employing the same solvent evaporation methods described in Example 1.

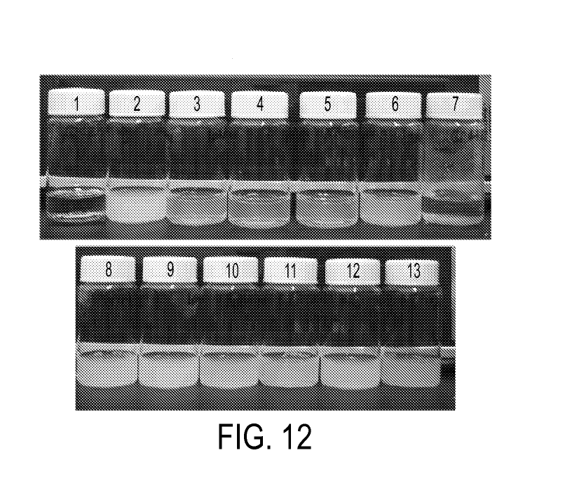

Characterization of Water-soluble WLNCs. Water-solubilization of the WLNCs was determined by visual inspection of the resulting solutions, as shown in FIG. 12. Solubilized nanocrystals were uniformly dispersed throughout the yellow solution, while unsolubilized nanocrystals deposited on the vial walls, as in the QD in water control. All functionalized AMs were capable of dispersing the WLNCs in the aqueous solutions. Solutions of the WLNCs dispersed in water using the phosphonic acid-terminal AMs (both propyl and butyl) and the phosphate-terminal AM appeared the most transparent of all dispersions (FIG. 12, samples 3-5). Solutions of the WLNCs in water using the sulfate and thiol-terminal AMs (FIG. 12, samples 6 and 11) had some transparency while all others appeared cloudy and turbid, similar to the carboxylic acid-terminal AM.

Turbidity. Turbidity was quantitatively determined using UV/Vis absorbance spectroscopy. WLNCs dispersed in water alone transmitted the most light across the tested wavelengths because all nanocrystals were adhered to the flask wall and, thus, the solution tested was only water. As a control, the turbidity of WLNCs dispersed in chloroform was tested. For these solutions, the only light transmitted was light not absorbed by the nanocrystals themselves. Consistent with the qualitative images in FIG. 12, solutions of the WLNCs dispersed in water using the phosphonic acid-terminal AMs (both propyl and butyl) and the phosphate-terminal AM transmitted the most light, with the solutions of phosphonic acid (butyl)-terminal AM-solubilized WLNCs transmitting the most light and, thus, being the least turbid. Solutions of the WLNCs dispersed in water using the sulfate and thiol-terminal AMs transmitted some light, as well, while all other functionalized AMs used to solubilize the WLNCs transmitted less than 20% light even at their peak transmittance. All polymer synthesized using Scheme A (above) are the polymers that subsequently solubilized the WLNCs yielding solutions with the least amount of turbidity. While this correlation between turbidity and synthetic methodology may be a coincidence, these results indicate that the synthetic method may influence turbidity of the functionalized-AMs and WLNC solutions.

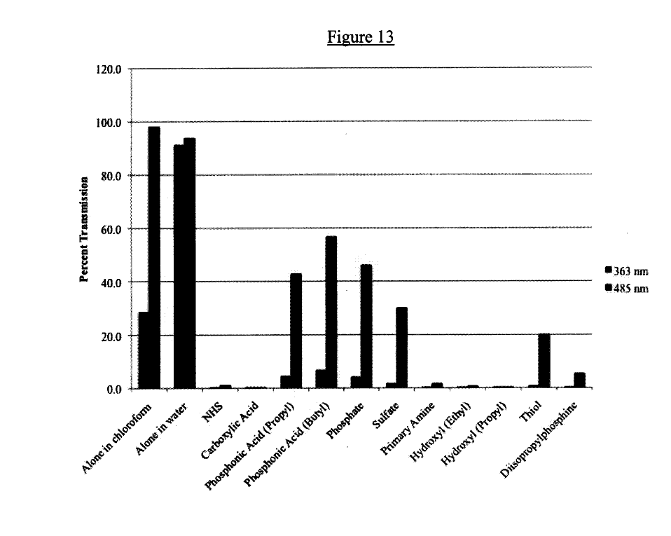

The percent transmittance of all samples at 363 and 485 nm are shown in FIG. 13. These wavelengths were chosen based upon the nanocrystals in dispersed chloroform; 363 nm is a peak for the percent transmittance and 485 nm is the wavelength at which maximum percent transmittance is obtained. The trends in FIG. 13 are the same as those discussed above, with nanocrystals dispersed in water using phosphonic acid (butyl)-terminal AM transmitting the most light, and the solutions of WLNCs with the phosphonic acid (propyl) and phosphate-terminal AMs transmitting 40-50% light at 485 nm. Other polymers yielding solutions of water-soluble WLNCs that transmitted light were the sulfate, thiol, and diisopropylphosphine-terminal AMs.

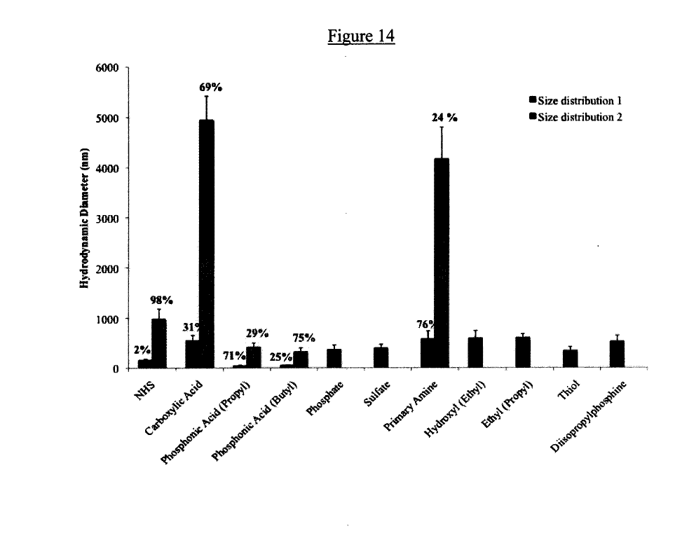

Assembly Sizes. Hydrodynamic diameters of the aqueous WLNC solutions were determined using dynamic light scattering. Graphical results are shown in FIG. 14 while the values are given in Table 4 (below).

TABLE 4
Hydrodynamic diameters observed for water-soluble assemblies
of WLNCs with the indicated functionalized AMs.

| | Size | Percentage | Size | Percentage |
|---|---|---|---|---|
| | Distribution | of | Distribution | of |
| | 1 (nm) | Volume (%) | 2 (nm) | Volume (%) |
| NHS | 159.3 | 2.4 | 976.6 | 97.6 |
| Carboxylic | 550.4 | 31.3 | 4943 | 68.7 |
| Acid | | | | |
| Phosphonic | 43.2 | 71.3 | 416.3 | 28.7 |
| Acid (Propyl) | | | | |
| Phosphonic | 49.3 | 25.4 | 318.6 | 74.6 |
| Acid (Butyl) | | | | |
| Phosphate | 363.2 | 100 | | |
| Sulfate | 390.9 | 100 | | |
| Primary | 575.7 | 75.6 | 4159 | 24.4 |
| Amine | | | | |
| Hydroxyl | 591.2 | 100 | | |
| (Ethyl) | | | | |
| Ethyl (Propyl) | 602.2 | 100 | | |
| Thiol | 338.3 | 100 | | |
| Diisopropyl- | 528.2 | 100 | | |
| phosphine | | | | |

The smallest assembly sizes were obtained using both phosphonic acid-terminal AMs, with one size distribution of 40-50 nm. However, the phosphonic acid (propyl)-terminal AM produced water-soluble WLNC solutions with the greatest percent by volume of the small size distribution (71% compared with only 25% for the phosphonic acid (butyl)-terminal AM-WLNC solutions). The other size distribution for each solution is consistent with the smallest size distributions (300-400 nm) observed for other AM-solubilized WLNC solutions using AMs synthesized by Scheme A (i.e. phosphate, sulfate, and thiol). For all other polymers, no assembly sizes smaller than 500 nm were observed.

As expected, this data is consistent with the turbidity data—the least turbid solutions contained assemblies of smaller sizes. Together, these data suggest a difference between the two synthetic methodologies.

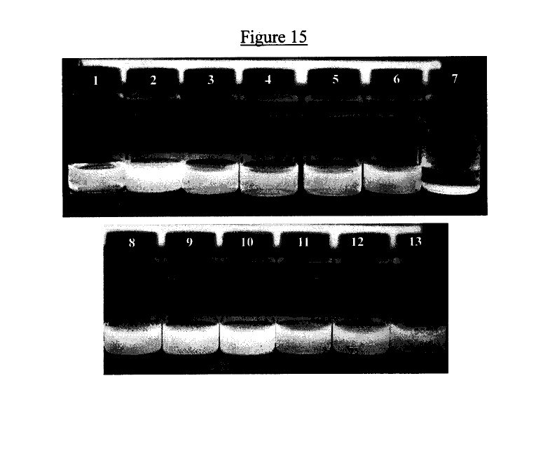

Qualitative Fluorescence Emission. Fluorescence emission was qualitatively determined by excitation with a long wavelength UV lamp at 365 nm. As shown in FIG. 15, all solutions except the WLNC dispersed in water only (sample 7) emit bright white light that is evenly dispersed throughout the solution, indicating successful solubilization.

For WLNC samples in water only, the WLNCs adhere to the vial walls, indicating a lack of aqueous solubilization of the hydrophobic nanocrystals. The nanocrystal adherence to the walls is observed by tipping the solutions on their sides. In comparison to the WLNCs dispersed in water using the sulfate-terminal AM in which the solution, even when tipped, emits evenly dispersed white-light, the WLNCs dispersed in water only have few nanocrystals dispersed in solution. Rather, the nanocrystals adhere to the flask.

Quantitative Fluorescence Emission. Quantitative fluorescence emission data for all samples imaged qualitatively was collected using fluorescence spectroscopy from 380-800 nm following excitation at 365 nm.

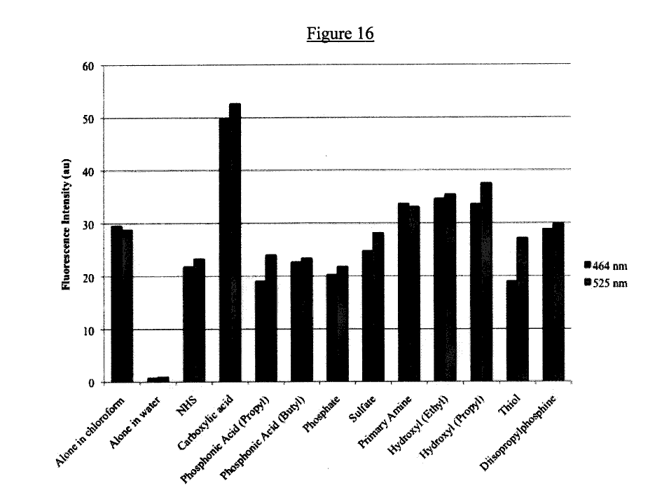

All solutions of WLNC, with the exception of the nanocrystals in water only, shared a similar fluorescence profile (i.e., broadband white light-emission and similar λₘₐₓ peaks) as the nanocrystals dissolved in chloroform at the same concentration. Overall, the WLNC water-solubilized utilizing the carboxylic acid-terminal AM had fluorescence intensity significantly greater than the nanocrystals in chloroform, consistent with the observations made in Example 1. To more easily compare the fluorescence intensities of the other samples, the fluorescence intensity at two wavelengths, 464 and 525 nm (the two λₘₐₓ peaks observed for the WLNCs in chloroform) are graphed for each sample in FIG. 16.

As shown in FIG. 16, only the data for the WLNCs water-solubilized by the primary amine-terminal AM follows the same trend for the WLNCs in chloroform in that the peak at 464 nm is slightly more intense than the emission peak at 525 nm. With respect to overall intensity, three of four of the functionalized AMs synthesized using Scheme B (the primary amine and both hydroxyl-terminal polymers), all water-solubilized the WLNCs in a manner that enhances their fluorescence intensity as compared with the WLNCs in chloroform at the same nanocrystal concentration. As discussed in Example 1, taken together with the size distributions, this data implies that solubilization of the nanocrystals into larger aggregates results in an overall enhancement of their fluorescence intensity. However, it should be considered that when 1cM was filtered with a 0.45 μm syringe in Example 1, the fluorescence intensity of the solutions significantly decreased. Given the similarities in this data, when the samples are similarly filtered, it is likely the same effect on fluorescence intensity would be observed.

The water-solubilization of the WLNCs using the diisopropylphosphine and sulfate-terminal AMs resulted in fluorescence similar to that of the nanocrystals in chloroform while the all other AMs resulted in fluorescence approximately 70% that of the nanocrystals in chloroform.

In summary, eleven polymers of varying functionalities were evaluated for their ability to water-solubilize WLNCs, retain nanoscale size and emit white light. Due to the differing physical states and solubilities of the amino-terminated starting materials, two synthetic methodologies were employed to synthesize polymers with the desired functionalities. In general, both turbidity and fluorescence data shows significant differences in polymer samples that, at first glance, correlate to the synthetic methods used. Based upon this data, it appears that both phosphonic acid-terminal AMs results in water-soluble WLNCs with the smallest sizes whilst maintaining white-light emission. In addition, based upon the results obtained in Example 1, the fluorescence emission of solutions of WLNCs in water with these polymers would be largely unaffected by filtration with 0.45 μm syringe filters due to the small-size assemblies.

### Example 3

### Synthesis of 1pM1cM

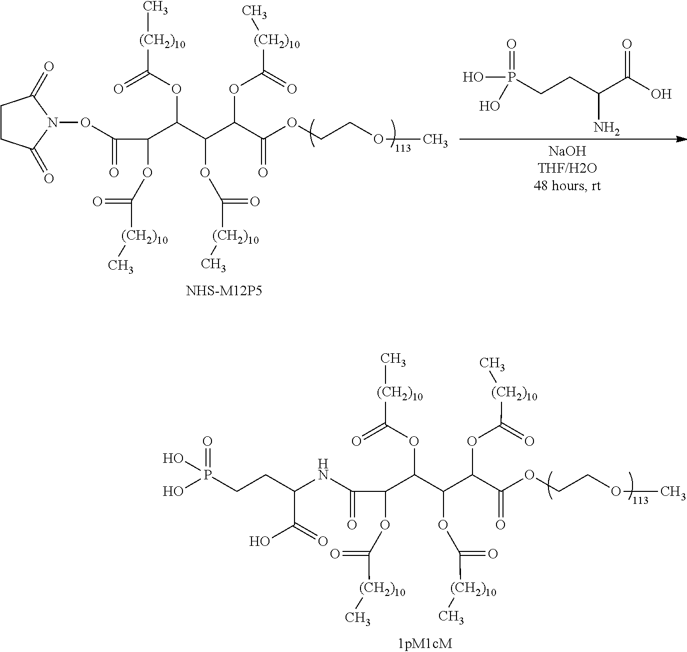

(±)-2-Amino-4-phosphonobutyric acid (36.8 mg, 0.201 mmol) was dissolved in H₂O (5 mL) and 1 M NaOH (0.3 mL) in the reaction flask. Note: the solid did not dissolve until the NaOH was added. In a separate vessel, NHS-M12P5 (0.3035 g, 0.050 mmol) was dissolved in THF (10 mL) at 37° C. in an incubator shaker. Upon checking the pH of the amine solution in the reaction flask, the pH was only 7-8, so 0.40 mL addition 1M NaOH solution was added to increase the pH to 10-12. The solution of NHS-M12P5 in THF was then added to the reaction flask over two minutes with stirring, at which point the solution turned yellow. The reaction was stirred for 48 hrs under ambient conditions. The THF was then removed by rotary evaporation (60° C. and lowest vacuum tolerated) and the pH of the resulting solution neutralized by adding 0.1 N HCl (5 mL), causing the solution to turn from yellow to clear. The solution was further acidified by adding 0.1 N HCl (8 mL). CH₂Cl₂ (50 mL) was then added and the solution stirred vigorously before being transferred to a separatory funnel and the CH₂Cl₂ layer removed. The aqueous portion was then washed with CH₂Cl₂ (2×-50 mL) and the combined organic portions washed with 50:50 Brine/H₂O (2×), dried over MgSO₄ and concentrated to a yellow oil. White solid was then precipitated from the oil in 4 CH₂Cl₂ (4 mL) by adding 10-fold diethyl ether in a 50 mL centrifuge tube. The tube was placed in the freezer for 15 min and then the solid collected by centrifuge and supernatant removed by decanting. The white solid was dried in the hood (24 hrs) and under high vacuum (48 hrs). NMR of the product in CDCl₃ showed no presence of NHS protons and a broad peak at ˜2 ppm, which could be the new methylenes. Yield=222.8 mg, 0.037 mmol (72.6%).

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An amphiphilic macromolecule of formula (I):
D-X—Y—Z—R₁ (I)
wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; R₁ is a polyether; R^{a} is a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alkyl.

2. The macromolecule of claim 1, wherein D is —C(═O)NR^{a}R^{b}; X is a polyol, Y is —C(═O)—, —C(═S)—, or is absent; Z is O, S or NH; R₁ is a polyether; R^{a} is a (C₁-C₁₀)alkyl group substituted with one or more —P(═O)(OH)₂ groups and optionally substituted with one or more carboxy COOH, or sulfo S(═O)₂OH groups; and R^{b} is H or (C₁-C₆)alkyl; wherein one or more hydroxy groups of the polyol are acylated with a fatty acid residue.

3. The macromolecule of claim 1, wherein R^{a} is a (C₁-C₁₀)alkyl group substituted with one or two —P(═O)(OH)₂ groups and optionally substituted with one or more carboxy COOH, or sulfo S(═O)₂OH groups.

4. The macromolecule of claim 1, wherein the polyol has from 2 carbons to 20 carbons.

5. The macromolecule of claim 1, wherein the polyol has from 2 to 10 hydroxy groups.

6. The macromolecule of claim 1, wherein the polyol is mucic acid, malic acid, citromalic acid, alkylmalic acid, hydroxy derivatives of glutaric acid, alkyl glutaric acids, tartaric acid, or citric acid.

7. The macromolecule of claim 6, wherein the polyol is mucic acid.

8. The macromolecule of claim 1 wherein the fatty acids comprise caprylic, capric, lauric, myristic, myristoleic, palmitic, palmitoleic, stearic, oleic, linoleic, arachidic, behenic, or erucic acid, or a mixture thereof.

9. The macromolecule of claim 1, wherein each fatty acid comprises 12 carbon atoms.

10. The macromolecule of claim 1, wherein Y is —C(═O)—; Z is O; and R₁ is —(CH₂CH₂O)₁₁₃—CH₃.

11. The macromolecule of claim 1, wherein D is:

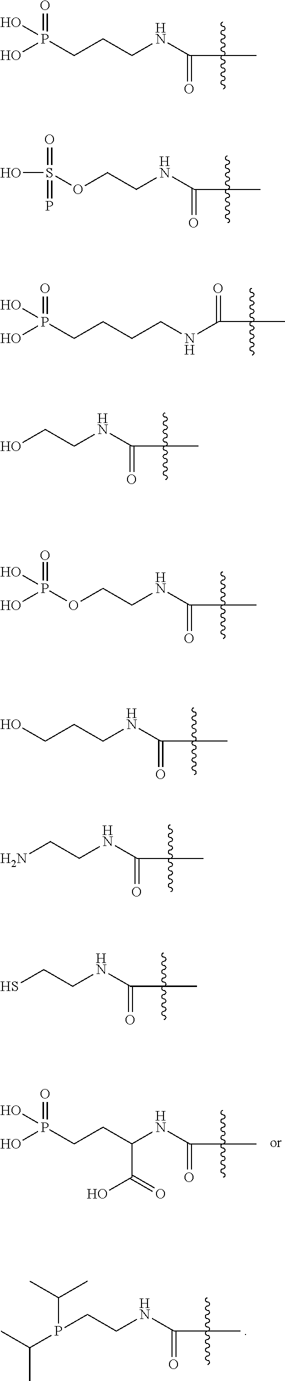

12. The macromolecule of claim 1, which is: 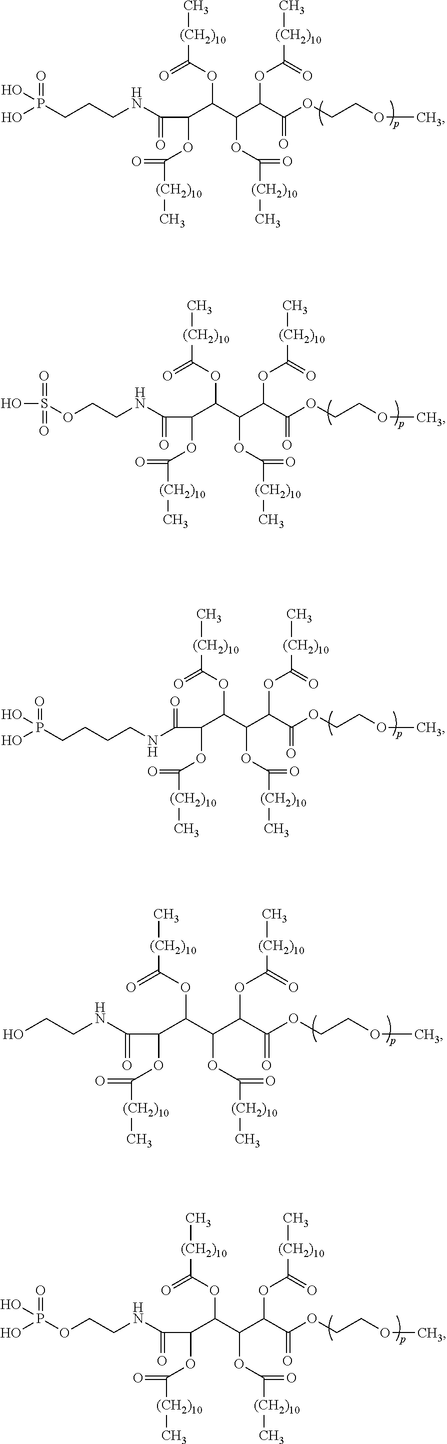 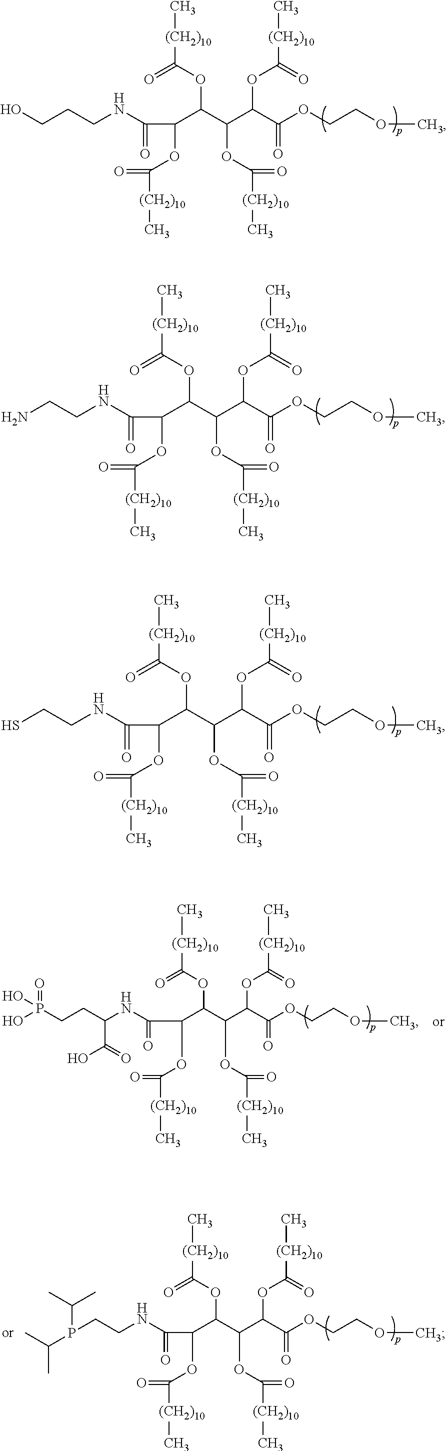
wherein p is about 113.

13. A composition comprising a plurality of macromolecules of formula (I), as described in claim 1, in a solvent.

14. A composition comprising a plurality of macromolecules of formula (I), as described in claim 1, in a solvent, wherein the macromolecule of formula (I) form one or more aggregate structures.

15. An aggregate structure formed by combining a plurality of macromolecules of formula (I), as described in claim 1, in a solvent; and allowing the macromolecules to form the aggregate.

16. An encapsulate comprising a molecule surrounded or partially surrounded by a plurality of macromolecules of formula (I) as described in claim 1 or residues thereof.

17. An encapsulate comprising a nanocrystal surrounded or partially surrounded by a plurality of macromolecules of formula (I):
D-X—Y—Z—R₁ (I)
or residues thereof, wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; each R₁ is independently a polyether; each R^{a} is independently a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alkyl.

18. The encapsulate of claim 17, wherein the nanocrystal is a light-emitting nanocrystal.

19. The encapsulate of claim 17, wherein the nanocrystal is a white-light-emitting nanocrystal.

20. The encapsulate of claim 17, wherein the nanocrystal is a quantum dot.

21. A pharmaceutical composition comprising an encapsulate, as described in claim 17, and a pharmaceutically acceptable carrier.

22. A method for delivering a nanocrystal to an animal comprising administering the composition of claim 21 to the animal.

23. An encapsulate formed by combining a) a plurality of macromolecules of formula (I):
D-X—Y—Z—R₁ (I)
wherein D is —C(═O)NR^{a}R^{b}, —C(═O)OR^{a}, or —C(═O)SR^{a}; X is a polyol, Y is —C(═O)—, —C(═S)—, or is absent; Z is O, S or NH; each R₁ is independently a polyether; each R^{a} is independently a (C₁-C₁₀)alkyl group substituted with one or more groups independently selected from —COOH, —P(═O)(OH)₂, —OP(═O)(OH)₂, —S(═O)₂(OH), —OSO₃H, —ON(═O)OH, —OH, —N(R^{b})₂, —SH, and —P(R^{c})₂, and optionally substituted with one or more —COOH, or sulfo S(═O)₂OH groups; each R^{b} is independently H or (C₁-C₆)alkyl; wherein one or more hydroxy groups of the polyol are acylated with a fatty acid residue; and each R^{c} is independently (C₁-C₆)alky; b) a nanocrystal, and c) a solvent; and allowing the macromolecules and nanocrystals to form the encapsulate.
